(19) United States
(12) Patent Application Publication
(10) Pub. No.: US 2004/0241835 A1
(43) Pub. Date: Dec. 2, 2004

(54) Dual axis bioreactor, system and method for growing cell or tissue cultures

(76) Inventors: Dietmar W. Hutmacher, Singapore (SG); Swee Hin Teoh, Singapore (SG); Manoja Ranawake, Balwyn (AU); Woon Shin Chong, Singapore (SG); Keng Soon Ting, Singapore (SG); Kay Chiang Chua, Singapore (SG); Than Myint, Singapore (SG); Chum Mok Puah, Singapore (SG); Toon Tien Foo, Singapore (SG); Jan-Thorsten Schantz, Singapore (SG)

Correspondence Address: DINSMORE & SHOHL LLP, Suite 500, One Dayton Centre, Dayton, OH, 45402-2050, US

(21) Appl. No.: 10/774,360

(22) Filed: Feb. 6, 2004

Related U.S. Application Data
(60) Provisional application No. 60/445,252, filed on Feb. 6, 2003.

(51) Int. Cl. C12M 3/02

(52) U.S. Cl. 435/298.2; 435/286.7; 435/394

(57) ABSTRACT

The invention relates to a continuous flow dual axis bioreactor (10) for growing three-dimensional cell or tissue cultures. The bioreactor (10) includes a chamber (12) adapted to contain a three-dimensional matrix for growing three-dimensional cell or tissue cultures. A rotatable plate (14) supports the chamber (12) and is rotatable about a vertical axis (16). The rotatable plate (14) is supported on an rotary L-shaped bracket (20) that rotates about a horizontal axis (22). Two multi-flow fluid connectors (32,34) are provided to prevent any pipes connecting the chamber to a feed source or a product tank from being entangled and to allow continuous flow to and from the bioreactor (10).

Two servo motors (86a,86b) are provided to respectively rotate the rotary plate (14) and the rotary L-shaped bracket 20 and thereby simultaneously rotate the chamber (12) about the vertical and horizontal axes (16,22) during growth of a three-dimensional cell or tissue culture.

A system and method for growing three-dimensional cell or tissue cultures is also disclosed.

### TECHNICAL FIELD

The present invention generally relates to a dual axis bioreactor for growing cell or tissue cultures and to a continuous flow dual axis bioreactor. The invention also generally relates to a method and system for growing cell or tissue cultures.

### BACKGROUND OF THE INVENTION

Defects in tissues resulting from disease or trauma have previously been healed through the regenerative process of wound healing. However, incomplete repair of the tissue may result when the defect is large, thereby resulting in fibrous scarring of the tissue. The fibrous scarring often possesses physical and mechanical properties that are inferior to that of non-scarred tissue.

Dramatic advances in the fields of biochemistry, cell and molecular biology, genetics, medicine, biomedical engineering and materials science have given rise to the cross-disciplinary field of tissue engineering, which uses synthetic or naturally derived, engineered scaffold/cell or scaffold/neotissue constructs for tissue regeneration. Ideally, tissue engineering aims to develop biological substitutes to solve the problem of organ and tissue deficiencies and provide medical implants. Bioreactors have been used to engineer cells and tissues.

In order to achieve optimal results in cell and tissue culture, the bioreactors should ideally operate to under conditions that are as close as possible to in vivo conditions. Difficulties have arisen with known bioreactors in that they have not provided a constant and regulatory supply of nutrition and removal of metabolic byproducts. Accordingly, it is desirable that bioreactor systems maintain an organotypic environment to maintain cellular differentiation and optimal function.

The multiplication of cells is most commonly performed in culture dishes with a static medium supplemented with growth serum. Although cells grown in culture dishes multiply quite well, they tend to loose their differentiation status and are therefore functionally different from naturally grown cells. This has been found to be the case with chondrocytes from cartilage. Isolated chondrocytes flatten and look more like fibroblastic mesenchymal/stromal cells. No basic cartilage extracellular matrix results.

Known cell and tissue cultures for cell and tissue repair have utilized mono-layers of cell and tissue. For example, in a skin defect reaching a lower layer of the dermis has been treated by debriding a slough or an abnormal granulation tissue, reconstructing a normal granulation tissue by covering the defect with an allogenic skin, wound dressings or the like, and then reconstructing skin by autologous split-thickness skin grafting. A disadvantage with this procedure is that skin is taken from non-defect area of the patient's skin and some scarring may remain at the graft site. Furthermore, in circumstances where a wound extends over a wide area, it is difficult to carry out autologous split-thickness skin grafting. To prevent or diminish scarring and to increase the healing time of damaged tissues, a regenerative process has been carried out in vitro by growing cell or tissue cultures on monolayers (ie two-dimensional cell or tissue cultures) on an artificial substrate that is bathed in nutrient medium. The nature of the substrate on which the monolayers grow may be solid, such as plastic, or semisolid gels, such as collagen or agar. Disposable plastics substrates are presently used in cell or tissue culture.

Although the growth of cells in two dimensions is suitable for studying cells in culture, it lacks the cell-cell and cell-matrix interactions that are characteristic of whole tissue in vivo. To grow cells that have the cell-cell and cell-matrix interactions that are characteristic of whole tissue in vivo, the cells should preferably be grown in three-dimensions. However, the growth of three-dimensional cells requires both physical and chemical signaling. Chemical signaling is generally realized through the constituents of the culture media. Physical signaling to grow cell or tissue cultures requires the use of bioreactors to grow the cell or tissue cultures in the substrates.

Current bioreactors for growing cell tissues are designed with only a single axis of rotation. These single axis rotating bioreactors subject the growing cells on a porous substrate to only a single force vector, thereby providing physical signaling only in the direction of that single force vector. Accordingly, the cells tend not to penetrate throughout the structure of the porous substrate and growth of three-dimensional cell or tissue cultures is inhibited.

Another disadvantage with some bioreactors for growing cell tissues is that they are designed to operate in batch or semi-batch mode.

It is an object of the invention to provide a bioreactor, a system or a method for growing cell or tissue cultures that overcome or at least ameliorate at least one of the disadvantages mentioned above.

Another object of the invention is to provide a continuous flow bioreactor for growing cell or tissue cultures.

A further object of the invention is to provide a bioreactor, a system or a method for growing cell or tissue cultures in vitro, that at least partially provide physical signaling in more than one force vector or flow vector or both.

A further object of the invention is to provide a bioreactor, a system or a method for growing three-dimensional cell or tissue cultures in vitro.

### SUMMARY OF THE INVENTION

According to a first aspect, the invention provides a dual axis bioreactor for growing cell or tissue cultures comprising:

a chamber for containing a cell or tissue culture and a culture medium for growing cell or tissue cultures;

a drive mechanism for rotating the chamber at a first speed about a first axis and at a second speed about a second axis, the second axis being substantially normal relative to the first axis, wherein the magnitude of the first speed and the second speed are independently variable of each other to thereby grow a cell or tissue culture within the chamber.

Suitably, the dual axis bioreactor further comprises:

a first rotatable member rotatable about the first axis and coupled to the chamber for rotating the chamber about the first axis; and

a second rotatable member rotatable about the second axis, the second rotatable member coupled to the chamber for rotating the chamber about the second axis.

Suitably, the dual axis bioreactor further comprises at least one fluid connector comprising:

a stationary casing;

a rotatable shaft mounted to the stationary casing, the shaft rotatable about a shaft axis in axial alignment with, or axially offset from, either the first or second axes; and

at least one fluidly sealed passage defined between the juncture of the stationary casing and the rotatable shaft and extending through the casing and the rotatable shaft, wherein the fluidly sealed passage allows passage of fluid from or to the chamber, or both, as the shaft rotates about the shaft axis.

Suitably, the dual axis bioreactor further comprises a heater element that is thermally coupled to the chamber for heating material within the chamber. The heater element may be disposed adjacent to an outer surface of the chamber.

In one embodiment, the dual axis bioreactor further comprises one or more detector elements for detecting a variable of the material within the chamber. The variable of the material within the chamber may be selected from the group consisting of: pH; temperature; dissolved oxygen content; and one or more combinations thereof.

In another embodiment the dual axis bioreactor further comprises a force detector for detecting the force applied the chamber as it rotates about the first axis or the second axis, or both.

Suitably, one fluidly sealed passage is provided in the fluid connector for passage of feed material to the chamber, and another fluidly sealed passage is provided in the fluid connector for passage of product material from the chamber.

Suitably, the dual axis bioreactor further comprises an adjustment mechanism provided on the first rotatable member or the second rotatable member for respectively adjusting the position of the chamber relative to the second axis or the first axis.

In a preferred embodiment, the drive mechanism includes at least one motor that is coupled to the first or second rotatable members, or both, by at least one drive shaft.

In a preferred embodiment, the drive mechanism includes:

a first motor coupled to the first rotatable member by an outer drive shaft having a hollow passage extending through its axis; and

a second motor coupled to the second rotatable member by an inner drive shaft disposed at least partly within the hollow passage of the outer drive shaft.

In a preferred embodiment, the drive mechanism includes:

a first motor coupled to the first rotatable member by a first drive shaft; and

a second motor disposed within, or on, the second rotatable member and coupled to the second rotatable member by a second drive shaft.

Suitably, the first and second motors are servo motors.

In a preferred embodiment, the drive shaft is coupled to the motor by a gear train for controlling the speed of rotation of the shaft.

In a preferred embodiment, the chamber further comprises a feed conduit for passage of feed media into the chamber and an outlet conduit for passage of product material from the chamber.

According to a second aspect of the invention, there is provided a method for growing cell or tissue cultures in vitro comprising the steps of:

(a) providing a chamber having a cell or tissue culture and a culture medium;

(b) rotating the chamber about a first axis at a first speed; and

(c) rotating the chamber about a second axis at a second speed, the second axis being substantially normal to the first axis and wherein the magnitude of the first speed and the second speed are independently variable of each other to thereby grow a cell or tissue culture.

According to a third aspect of the invention, there is provided a system for growing cell or tissue cultures in vitro comprising:

a bioreactor comprising

a chamber for containing a cell or tissue culture and a culture medium for growing cell or tissue cultures;

a drive mechanism for rotating the chamber at a first speed about a first axis and at a second speed about a second axis, the second axis being substantially normal relative to the first axis; and

a controller for controlling the operation of the drive mechanism, wherein the magnitude of the first speed and the second speed are independently variable of each other to thereby grow a cell or tissue culture within the chamber.

According to a fourth aspect of the invention, there is provided a continuous flow dual axis bioreactor for growing cell or tissue cultures comprising:

a chamber for containing a cell or tissue culture and a culture medium;

a first rotatable member rotatable about a first axis, the first rotatable member coupled to the chamber for rotating the chamber about the first axis in use;

a second rotatable member rotatable about a second axis, the second axis being substantially normal relative to the first axis, the second rotatable member coupled to the chamber for rotating the chamber about the second axis;

a drive mechanism for rotating the first rotatable member at a first speed about the first axis and the second rotatable member at a second speed about the second axis, wherein the magnitude of the first speed and the second speed are independently variable of each other to thereby grow a cell or tissue culture within the chamber; and

a fluid connector for providing fluid material passage to and from the chamber.

According to a fifth aspect of the invention, there is provided a cell or tissue culture when grown in vitro by the method of the second aspect.

According to a fifth aspect of the invention, there is provided a three-dimensional cell or tissue culture when grown in vitro by the method of the second aspect.

### Definitions

The following words and terms used herein shall have the meaning indicated:

The word “fluid” and the term “fluid material” are to be interpreted broadly to include not only liquid and gas phase materials but also slurries that comprise solid or semi-solid material suspended in a liquid phase.

The term “feed material” is to be interpreted broadly to include a liquid phase or a gas phase material or a slurry that comprises solids or semi-solids suspended in a liquid phase, and combinations of one or more phases thereof, which is used to facilitate the growth of cell or tissue cultures.

The words “culture medium” or “culture media”: are to be interpreted broadly to include any medium that facilitates the growth of cell and tissues.

The term “product material” is to be interpreted broadly to include a liquid phase or a gas phase material or a slurry that comprises solids suspended in a liquid phase, and combinations of one or more phases thereof, which includes one or more reactant products, by-products or intermediate products produced as a result of the growth of cell or tissue cultures.

The term “substantially normal” and grammatical variations thereof, throughout the specification and the claims is to be interpreted broadly to include the second axis being perpendicular relative to the first axis and the second axis and also including anywhere within an arc covering the range of 60° to 120° relative to the first axis.

The terms “three-dimensional matrix” or “three-dimensional matrices”: are to be interpreted broadly to include any (a) any material and/or shape, including gels, beads, porous meshes, scaffolds, that have three dimensions and which allow cells to attach to it (or can be modified to allow cells to attach to it); and (b) allows cells to grow in more than one layer.

The words “matrix” or “matrices”: are to be interpreted broadly to include any (a) any material and/or shape, including gels, beads, porous meshes, scaffolds, which allow cells to attach to it (or can be modified to allow cells to attach to it).

### BRIEF DESCRIPTION OF THE DRAWINGS

Preferred embodiments of the invention will now be described with reference to the following drawings.

FIG. 1 shows a perspective view of a dual axis bioreactor apparatus in accordance with one preferred embodiment.

FIG. 2 shows a side view of the dual axis bioreactor apparatus of FIG. 1.

FIG. 2a shows a partial cross-sectional view of the dual axis bioreactor shown in FIG. 1 in the plane shown by the direction of arrows AA.

FIG. 3 shows a top view of the dual axis bioreactor apparatus of FIG. 1.

FIG. 4 shows a detailed perspective view of the chamber assembled to the dual axis bioreactor apparatus of FIG. 1.

FIG. 5 shows a more detailed perspective view of the chamber of to the dual axis bioreactor apparatus of FIG. 1.

FIG. 6 shows a perspective view of a pair of surgical needles mounted to the clamp cover of the chamber of the dual axis bioreactor apparatus of FIG. 1.

FIG. 7 shows a detailed perspective view of an adjustment mechanism mounted to the dual axis bioreactor apparatus of FIG. 1.

FIG. 8 shows a perspective view of the adjustment of FIG. 13 disassembled from the dual axis bioreactor.

FIG. 9 shows a perspective view of a pipe connector assembled on the dual axis bioreactor shown in FIG. 1.

FIG. 10 shows a perspective view of the pipe connector of FIG. 9.

FIG. 11 shows an end view of the pipe connector of FIG. 10.

FIG. 12 shows a section view of the pipe connector taken along the arrow lines W-W shown in FIG. 11.

FIG. 13 shows cross-section view of the multi-flow pipe connector taken along the arrow lines Y-Y shown in FIG. 11.

FIG. 14 shows a perspective sectional view of the pipe connector of FIG. 10.

FIG. 15 shows a perspective view of the dual axial drive shafts of a second embodiment of the dual axis bioreactor, with the pipe connectors removed.

FIG. 16 shows a perspective view of the drive assembly of FIG. 15.

FIG. 17 shows a top view of the drive assembly of FIG. 16.

FIG. 18 shows an end view of the drive assembly of FIG. 16.

FIG. 19 shows a cross sectional view of the drive assembly taken along the arrow lines A-A of FIG. 17.

FIG. 20 shows an exploded perspective view of the drive assembly of the dual axis bioreactor shown in FIG. 15.

FIG. 21 shows a side cross-sectional view of the dual axial drive shaft of a second embodiment of the dual axis bioreactor of FIG. 15.

FIG. 22 shows a top view of the dual axial drive shaft of a second embodiment of the dual axis bioreactor of FIG. 15.

FIG. 23 shows a schematic diagram of a system for growing cell or tissue cultures in vitro using the bioreactor of FIG. 1.

FIG. 24 shows a schematic diagram of an alternative system to the system of FIG. 23 for growing cell or tissue cultures in vitro using the bioreactor of FIG. 1

FIG. 25 shows a schematic of a control system for the system of FIG. 23.

FIG. 26 shows a front view of an alternative dual axis bioreactor apparatus in accordance with another preferred embodiment.

FIG. 27 shows a perspective view of the dual axis bioreactor apparatus of FIG. 26.

FIG. 28 shows a side view of the dual axis bioreactor apparatus of FIG. 26.

FIG. 29 schematically shows the steps of a method that is used to grow a three-dimensional skin culture in vitro using the system of FIG. 23.

FIG. 30 shows an SEM micrograph of goat chondrocytes seeded onto a 3D ear shaped scaffold, which was incubated in a static environment in accordance with the prior art.

FIG. 31 shows an SEM micrograph of goat chondrocytes seeded onto a 3D ear shaped scaffold, which was incubated in a bioreactor that rotated about a single axis of rotation in accordance with the prior art.

FIG. 32 shows an SEM micrograph of goat chondrocytes seeded onto a 3D ear shaped scaffold, which was incubated in the bioreactor of FIG. 1 in accordance with the present invention.

FIG. 33 shows a bar graph of cell metabolic activity of the goat chondrocytes cells grown statically, in a single rotating axis bioreactor and in the bioreactor of FIG. 1.

### DETAILED DESCRIPTION OF PREFERRED EMBODIMENTS OF THE INVENTION

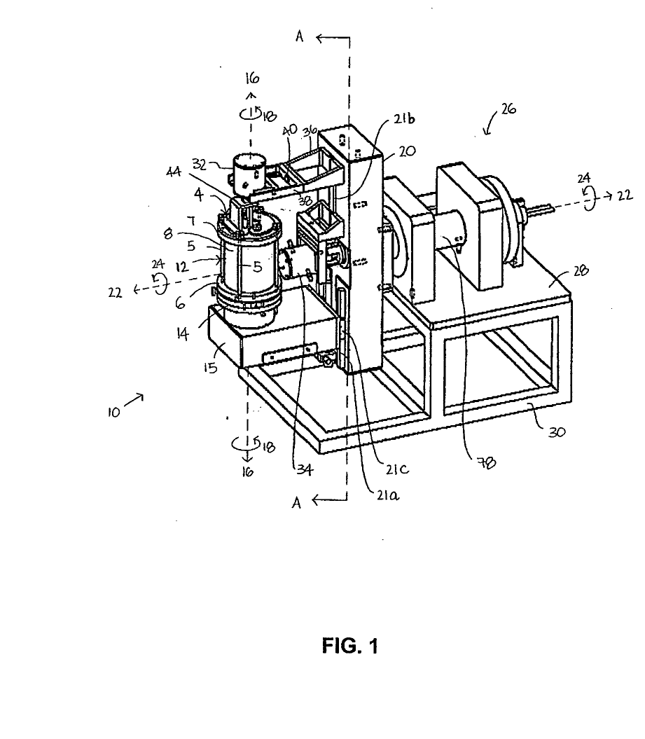

FIG. 1 shows a perspective view of a first preferred embodiment of a dual axis bioreactor 10 that is used to grow cell or tissue cultures. The bioreactor 10 includes a chamber 12 for containing a cell or tissue culture and a culture medium for growing cell or tissue cultures in use. The bioreactor 10 also includes a drive mechanism 26 for rotating the chamber 12 at a first speed about a first vertical axis 16 and at a second speed about a second horizontal axis 22. The horizontal axis is normal relative to the vertical axis. As will be described further below, in use, the magnitude of the first speed and the second speed are independently variable of each other to thereby grow a cell or tissue culture within the chamber.

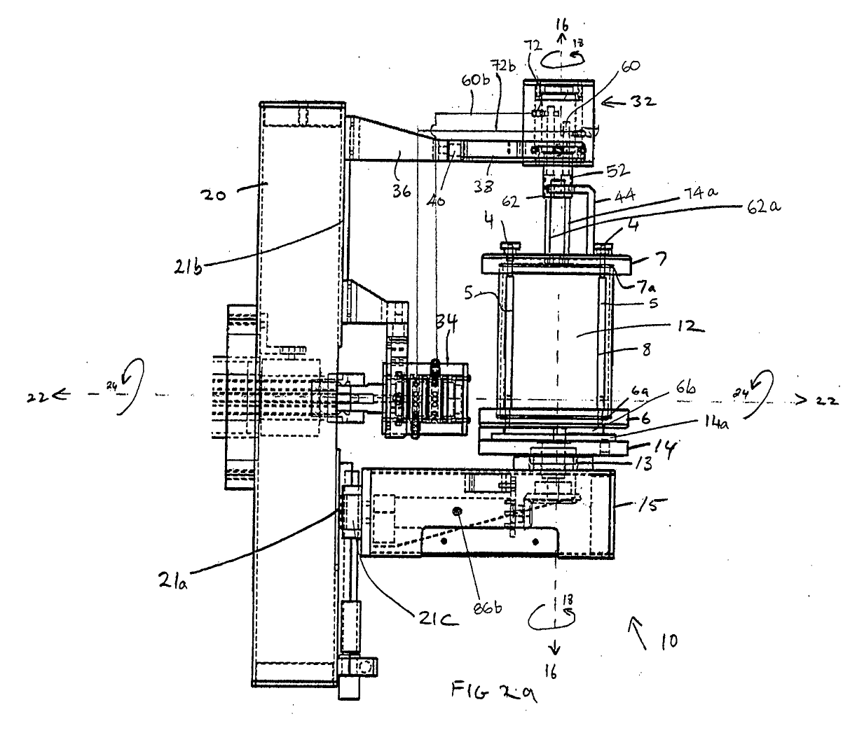

Referring to FIGS. 1, 2, 2a, 3, the chamber 12 may be provided with a three-dimensional matrix (not shown) when growing three-dimensional cell or tissue cultures as will be described further below. The chamber 12 includes a glass tube 8 having two open ends that are respectively clamped between a top flange 7 and a bottom flange 6 by four evenly spaced rods 5 fixed by a knurled locking nut and bolt arrangement 4.

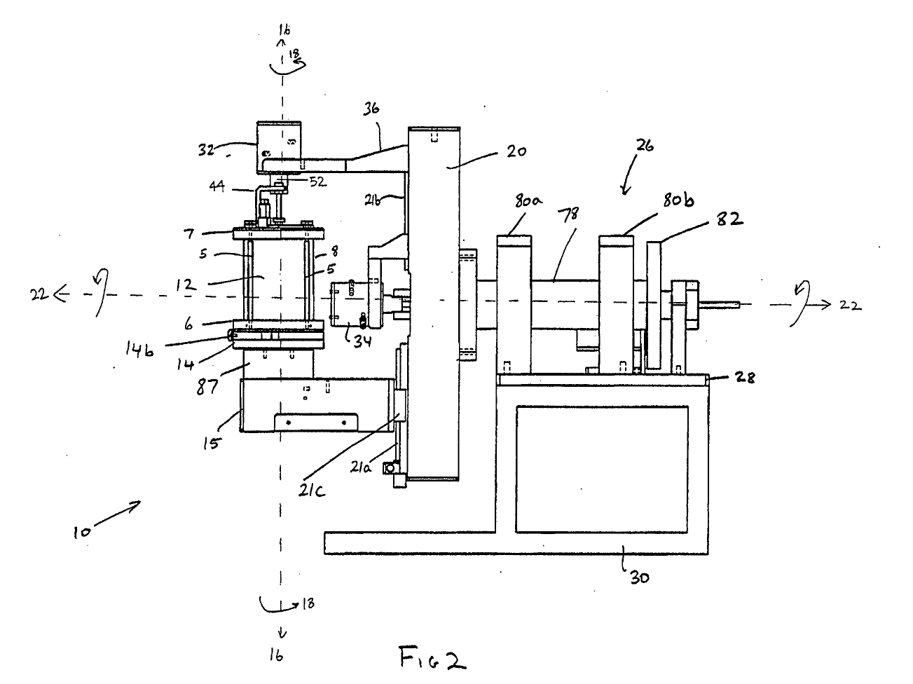

In this embodiment, the top flange 7 and the bottom flange 6 are manufactured from stainless steel. Referring to FIGS. 2a, it can be seen that the chamber 12 further includes seals 6a and 7a respectively provided between the ends of the glass tube 8 and between the bottom flange 6 and top flange 7.

Referring to FIG. 5, which shows a detailed view of the chamber 12, removal plugs (not shown) are provided in the top flange 7 (refer to FIG. 15) for insertion and retrieval of fluid material within the chamber 10 that is used to grow the cell tissue cultures. The holes into which the removal plugs are inserted are used as conduits for respectively allowing passage of fluid material into and out of the chamber 12.

In this embodiment, two stainless steel tubes 7d,7e are provided to extend through the conduits of the flange 7 and thereby respectively provide a conduit for passage of fluid material into and out of the chamber 12. The flange 7 is also provided with detectors for detecting process variables associated with the fluid material within chamber 12. The detectors in this embodiment include a temperature sensor 7f for measuring the temperature of the fluid material, dissolved oxygen sensor 7g for measuring the dissolved oxygen content of the fluid material, and a pH sensor 7h for measuring the pH of the fluid material.

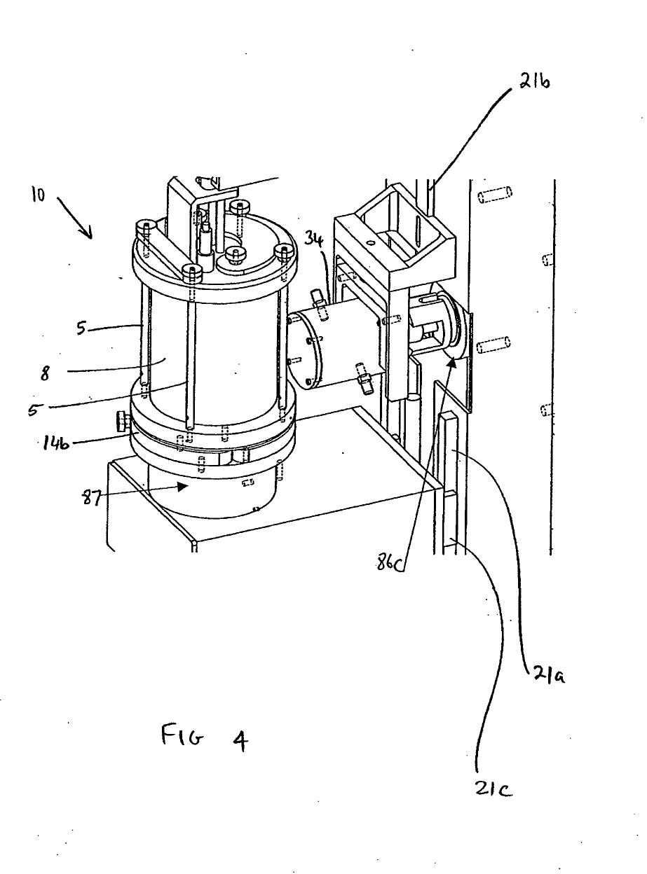

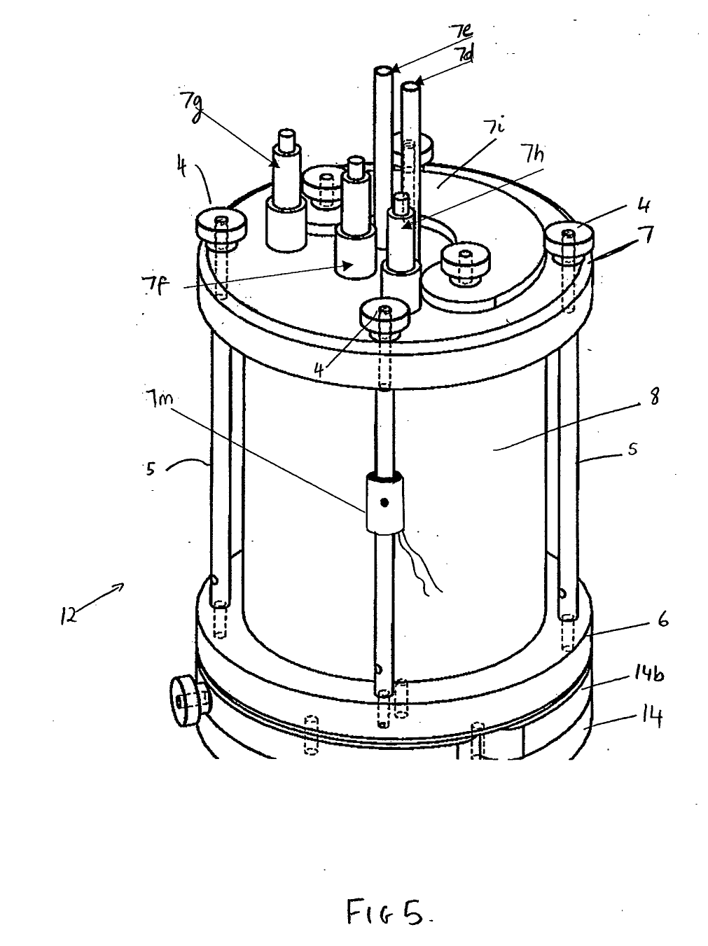

The flange 7 is also provided with a chamber cover 7i for introducing a three dimensional matrix or a scaffold into the chamber 12. A more detailed view of the chamber cover 7i can be seen in FIG. 6, which shows a perspective view of the chamber cover 7i when disassembled from the chamber 12. The chamber cover 7i includes a mount for retaining a scaffold in the form of a pair of surgical needles 7j,7k, which are used to impale a three-dimensional matrix onto in use. The three-dimensional matrix can be any kind of porous scaffold and is used to provide an attachment structure for the grows of three-dimensional cell cultures and tissues thereon in use.

Referring to FIG. 5, the flange 7 also has a force detector 7m, which attached to its surface for detecting centripetal and centrifugal forces applied to the chamber 12 as it rotated about vertical axis 16 and horizontal axis 22.

Referring now again to FIGS. 1-2,2a and 3, the bioreactor 10 also includes a first rotatable member in the form of rotary plate 14 that is mounted on a rotor 13 of (refer to FIG. 2a) a servo-motor 86b. The rotary plate 14 is rotatable about a vertical axis as shown generally by dashed arrow 16, in the direction of arrow 18. It should be realized that in other embodiments, the rotary plate 14 may rotate about the vertical axis in an opposite direction to the direction of arrow 18. The rotary plate 14 is clamped by the bottom flange 6 of the chamber 12 so that when in use, the rotary plate 14 rotates the chamber 12 about the vertical axis 16.

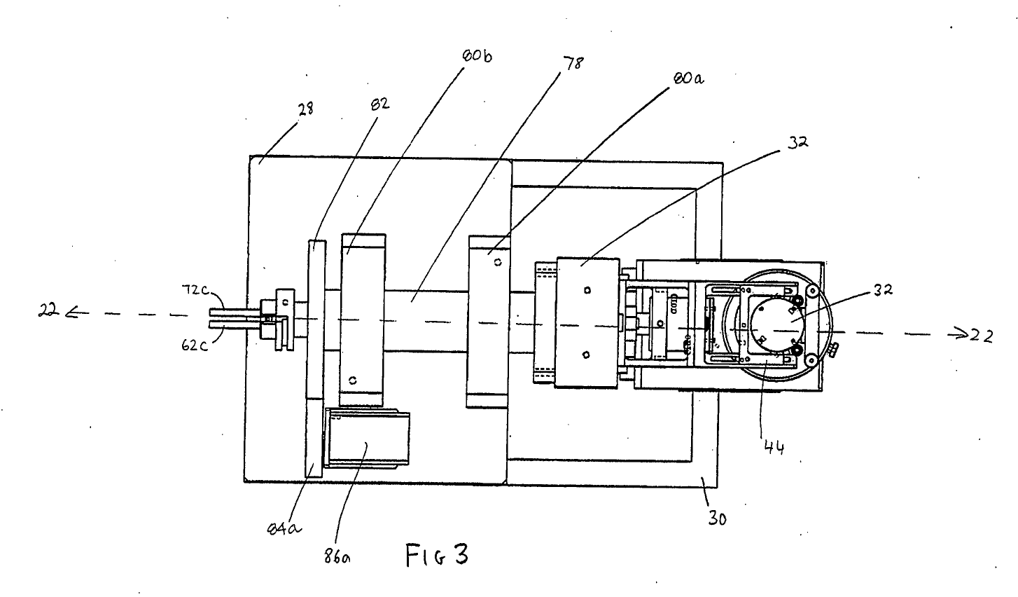

Referring now to FIG. 2a, the bioreactor also includes a heater element in the form of two heating cartridges 6b mounted within rotary plate 14. Bracket 14a of flange 6 sits on rotary plate 14 and is locked thereto by brace 14b (refer to FIGS. 4-5). The heating cartridges 6b are thermostatically controlled by a controller for maintaining the temperature within the chamber 12 during use. The heating cartridges 6b within heater plate 14a is provided adjacent to the bottom of the chamber 12 to effect efficient heating of the fluid material within the chamber 12.

The bioreactor 10 includes a second rotatable member in the form of rotary L-shaped bracket 20. The L-shaped bracket 20 includes a horizontal support arm 15 having a longitudinal axis that is in alignment with, but offset from, the horizontal axis 22. Rotary L-shaped bracket 20 is rotatable about a horizontal axis as shown generally by dashed arrow line 22, in the direction of arrow 24. It should be realized that in other embodiments, the rotary L-shaped bracket 20 may rotate about the horizontal axis 22 in an opposite direction to the direction of arrow 24.

In this embodiment, the horizontal axis 22 is at a right angle relative to the vertical axis 16. It should be appreciated however, that the vertical axis 16 may not be at a right angle relative to the horizontal axis 22 but may extend anywhere within an arc covering the range of 60° to 120° relative to the horizontal axis.

Referring to FIG. 2a, the rotary drive 13 is mounted to the support arm 15 of the rotary L-shaped bracket 20, and provides a support for the rotary plate 14 so that, as will be described further below, the rotary L-shaped bracket 20 rotates the chamber 12 about the horizontal axis 22.

The drive mechanism 26 is mechanically coupled to the rotary L-shaped bracket 20 and the rotary drive 13 to simultaneously rotate the chamber 12 about the horizontal axis 22 and the vertical axis 16 and thereby subject a growing cell or tissue culture within the chamber 12 to two force vectors in order to propagate a three-dimensional cell or tissue culture.

In other embodiments, it should be realized that periodic or sequential rotation of the rotary L-shaped bracket 20 and the rotary drive 13 may occur rather than simultaneous rotation when growing cell or tissue cultures.

The drive mechanism 26 is supported on a base plate 28, which is connected to frame 30. The frame 30 is shaped such that the base plate 28 is at a height from the ground such that it is sufficient to allow the rotary L-shaped bracket 20 to rotate about the horizontal axis 22 without interference.

Referring to FIGS. 1-2,2a,7 and 8, tracks 21a, 21b are provided on the rotary L-shaped bracket 20. The tracks 21a, 21b are shaped such that they allow guides 21c that are provided on the support arm 15 and the bracket 36 to travel thereon. The guides are provided with a locking mechanism 21h that locks the support arm 15 and the bracket 36 in a desired position during use.

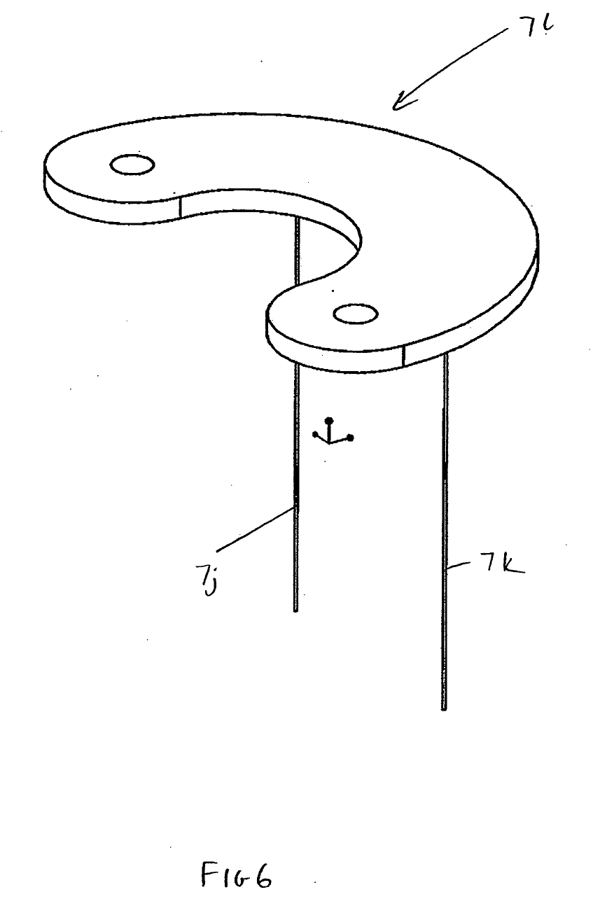

Referring now to FIG. 8, there is shown a view of two tracks 21a and guides 21c when disassembled from the dual axis bioreactor 10. The guides 21c are mounted to a plate 21e that has two extending arms 21d extending from the face of the plate 21e to form a yoke that mounts to the support arm 15.

A lead screw shaft 21f is disposed between the tracks 21A and is connected to the plate 21e by a lead screw nut 21g. It will be appreciated that the support arm 15 and the bracket 36 are moveable along the vertical axis 16 by actuating the lead screw nut 21g along the lead screw shaft 21f to so that the position of the chamber 12 and the connector 32 can be varied with reference to the horizontal axis 22. Accordingly, a user is able to change the centrifugal and centripetal forces acting on the growing cells or tissues within the chamber 12.

Referring again to FIGS. 1-3, the drive mechanism 26 of the bioreactor 10, includes a main drive shaft 78, which extends through two mounting plates 80a,80b attached to base plate 28. The drive shaft 78 extends through the mounting plate 80a and connects to the rotary L-shaped bracket 20 to rotate the arm, in use, about the horizontal axis 22.

The drive mechanism 26 also includes a gear train 82 provided adjacent to mounting plate 80b. The gear train 82 is driven by a rotor 84a that is actuated by servo motors 86a mounted on base plate 28. The servo motors 86a,86b are operated by a controller, as will be described further below. The servo motor 86a drives the drive shaft 78 and hence the rotary L-shaped bracket 20. The drive mechanism also includes the servo motor 86b (refer to FIG. 2a) that is mounted within support arm 15 and has a rotary drive that supports rotary plate 14.

Referring to FIG. 4, slip-rings are also associated with both servo-motors 86a,86b. The slip-ring associated with the servo-motor 86b is housed within housing 87 and a slip-ring 86c. The slip-rings are provided for command signals to be sent to each of the servo-motors 86a,86b and for data transfer between the servo-motors 86a,86b and the controllers. The slip-ring housed within housing 87 can also be used to provide data transfer from the temperature sensor 7f, dissolved oxygen sensor 7g, pH sensor 7h and force detector 7m mounted to the flange 7. The servo motors 86a,86b are also provided with encoders to monitor the position of the rotors and the encoders send data through the respective slip-rings for control over the bioreactor 10.

The servo-motors 86a and 86b are both brushless servo-motors. The servo-motor 86a is able to operate the rotary L-shaped bracket 20 at a speed in the range between 1 to 80 rpm and at a continuous torque of 5 Nm with a peak of 10 Nm. The servo-motor 86b is able to operate the rotary arm 14 at a speed in the range between 1 to 80 rpm and at a continuous torque of 1 Nm with a peak of 2 Nm.

As can be seen in FIGS. 1,2 and 2a, the bioreactor 10 includes two fluid connectors in the form of pipe connectors 32,34. The pipe connectors 32,34 are “multi-flow” pipe connectors in that they allow passage of fluid material to and from the chamber 12 during use and are provided to prevent entanglement of pipes supplying feed material from a support fermenter to the chamber 12. As will be explained further below, the pipe connectors 32,34 enable the bioreactor 10 to function as a continuous flow bioreactor.

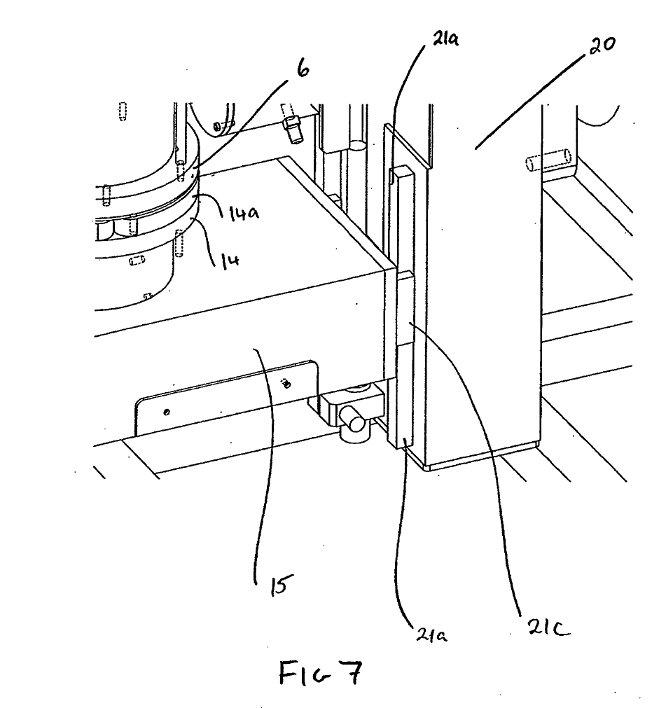

Referring to FIG. 9, there is shown a detailed perspective view of the pipe connector 32 mounted above the chamber 12 by a yoke 38. The yoke 38 is attached to a bracket 36 by a universal joint 40. The bracket 36 is attached to rotary L-shaped bracket 20. The universal joint 40 allows the connector 32 mounted to the yoke 38 to rotate about a second, horizontal axis 42 that is offset from, but parallel to, the horizontal axis 22. The pipe connector 32 is also mounted to the yoke by a universal joint (not shown) to allow the pipe connector to swivel. The universal joint 40 also provides minimal seal degradation over prolonged use. The pipe connector 32 is attached to the top flange 7 of the chamber 12 by a bracket 44.

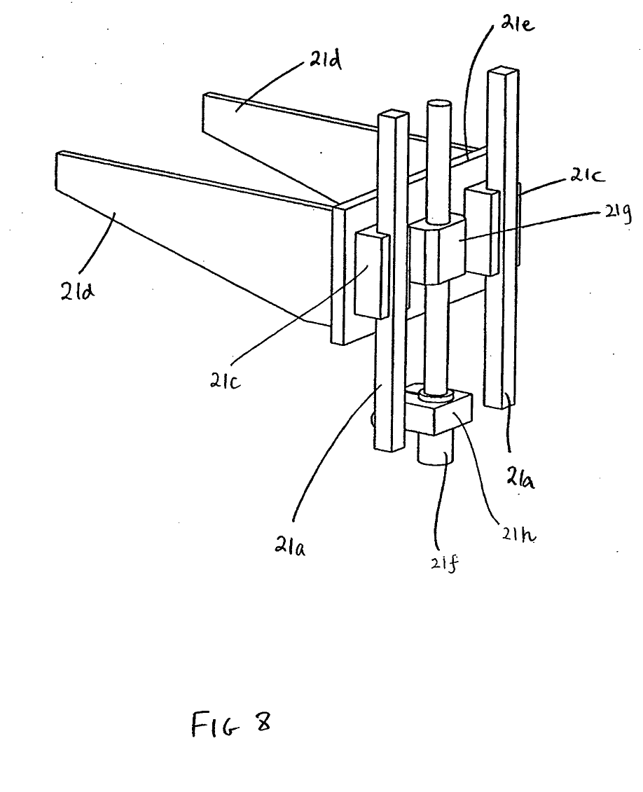

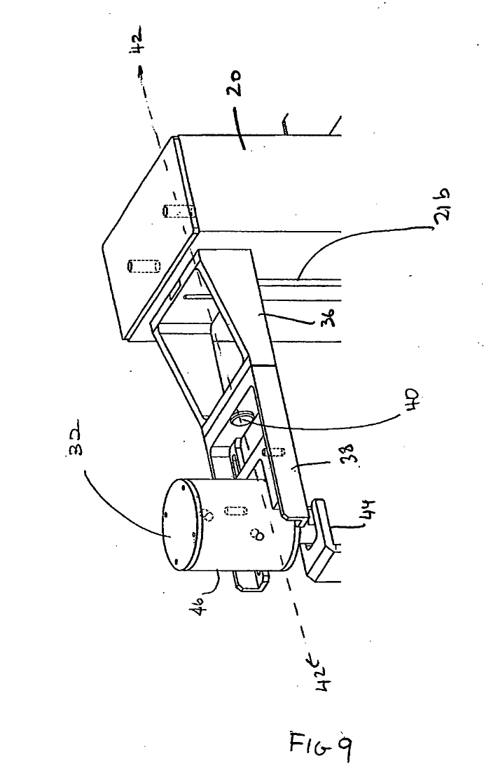

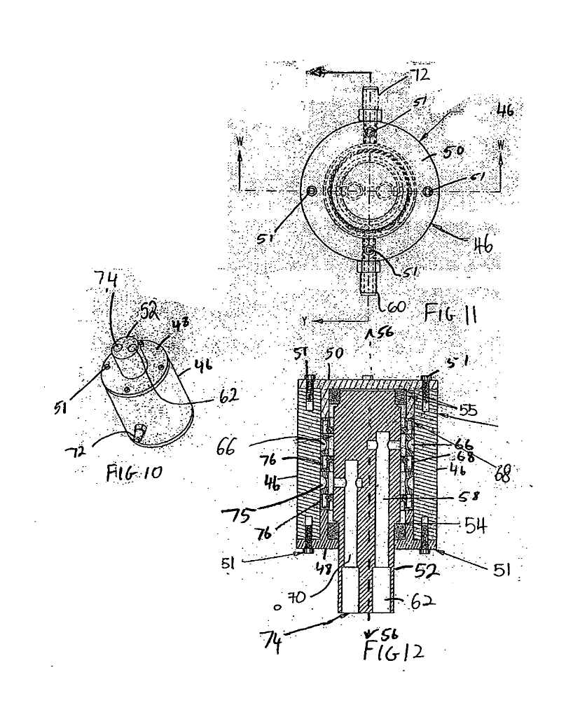

The components of the pipe connector 32 will be discussed in detail by referring to FIGS. 10 to 14. It should be understood that the components of the pipe connector 34 are identical to that of pipe connector 32 and the detailed component description of pipe connector 34 is provided merely for convenience.

The pipe connector 32 includes stationary casing in the form of tubular casing 46 having two open ends. The open ends of the casing 46 are clamped between a front flange 48 and a rear flange 50 by a locking nut and bolt arrangement 51.

The pipe connector 32 further includes a rotatable shaft 52 mounted to the casing 46 and extending from the casing 46 via a hole provided in the front flange 48. A front ball bearing 54 is provided adjacent to the inner side of the front flange 48 and a rear ball bearing 55 is provided adjacent to the inner side of the rear flange 50 to allow the shaft 52 to rotate about the shaft axis shown by dashed arrow line 56 in FIG. 12.

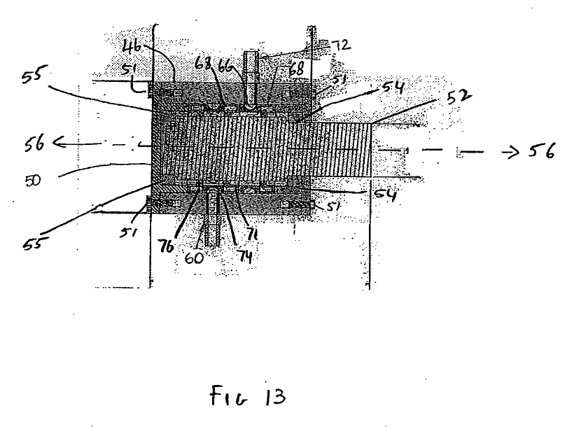

The pipe connector 32 includes a feed material passage in the form of feed conduit 58 extending between inlet nipple 60 (refer to FIG. 13 and FIG. 14) and outlet conduit 62 (refer to FIG. 12 and FIG. 14). The outlet conduit 62 has internal threads to allow for connection with pipe 72a shown in FIG. 2a. The pipe 72a enables the outlet conduit 62 to be in fluid communication with the chamber 12.

As shown in FIGS. 12-14, an inflow cavity 66 extends around the inner wall of the tubular casing 46 and between the shaft 52 and is bound on adjacent sides by a seal in the form of spring-loaded rubber lip oil seals 68.

As the inflow cavity 66 extends around the circumference of the shaft 52, as the shaft 52 rotates about the shaft axis 56, the feed conduit 58 is always in fluid communication with both the inlet nipple 60 and the outlet conduit 62.

The pipe connector 32 includes a product material passage in the form of product conduit 70 extending between outlet nipple 72 (refer to FIG. 13) and inlet conduit 74 (refer to FIG. 12). The inlet conduit 74 has internal threads to allow for connection with a pipe (not shown) that is able to be inserted into a hole provided in the top flange 7 to allow the inlet conduit 74 to be in fluid communication with the chamber 12. In FIG. 12 and FIG. 13, an outflow cavity 75 extends around and between the inner wall of the tubular casing 46 and the shaft 52 and is bound on adjacent sides by another pair of seals in the form of spring-loaded rubber lip oil seals 76. Accordingly, the outflow cavity 75 is fluidly sealed from the inflow cavity 68.

As the outflow cavity 75 extends around the circumference of the shaft 52, as the shaft 52 rotates about the shaft axis 56, the product conduit 70 is always in fluid communication with both the outlet nipple 72 and the inlet conduit 74.

In use, the inlet nipple 60 can be attached to a material feed source, such as a fermenter, to supply feed material to the outlet conduit and ultimately to the chamber 12. Furthermore, the pipe (not shown) connected to the inlet conduit 74 allows product material to be removed from the chamber 12 and transfers it to a product material tank (not shown).

Referring again now to FIGS. 1-3, in this embodiment the shaft axis 56 of pipe connector 32 is co-axial with the vertical axis 16. As the rotary plate rotates about the vertical axis 16, the bracket 44, which is attached to flange 7, engages rotary shaft 52 causing it to turn about the vertical axis 18 in a period that is synchronous with the rotation of the chamber 12 about the vertical axis 16. Accordingly, it will be appreciated that entanglement of the pipes 60a,74a will not occur as a result of this synchronous rotation.

It will be appreciated that the pipe connector 32 allows the bioreactor 10 to function as a continuous flow bioreactor. The ability of the bioreactor 10 to function continuously provides enhanced throughput compared to operating in batch mode. This is particularly advantageous in industrial scale applications where the enhanced throughput enables the realization of efficiencies that may not be achievable in batch operation. Furthermore, the pipe connectors allow continuous re-circulation of media to and from the chamber 12 and a support fermenter as will be described further below.

Referring to FIGS. 2a, it can be seen that inlet nipple 60 and outlet nipple 72 of pipe connector 34 are respectively coupled to pipes 60b,72b. Pipes 60b,72b are respectively coupled to like nipples provided on pipe connector 34. This allows like outlet nipples on fluid connector 34 to be connected to inlet and outlet pipe lines in for continuous or re-circulatory flow of material to and from the chamber 12 as the chamber rotates about the horizontal axis 22 and vertical axis 16.

FIG. 15 a second preferred embodiment of a dual axis bioreactor 10′. The numbered features of the bioreactor 10′ are the same as that of bioreactor 10 but are shown with the prime symbol (′)for convenience and will not be described again here. The pipe connectors 32′,34′ are not shown in the figures for convenience. The drive mechanism 26′ is different to the drive mechanism 26 of bioreactor 10, because servo-motor 86b′ is not located in support arm 15 but is located on base plate 28′.

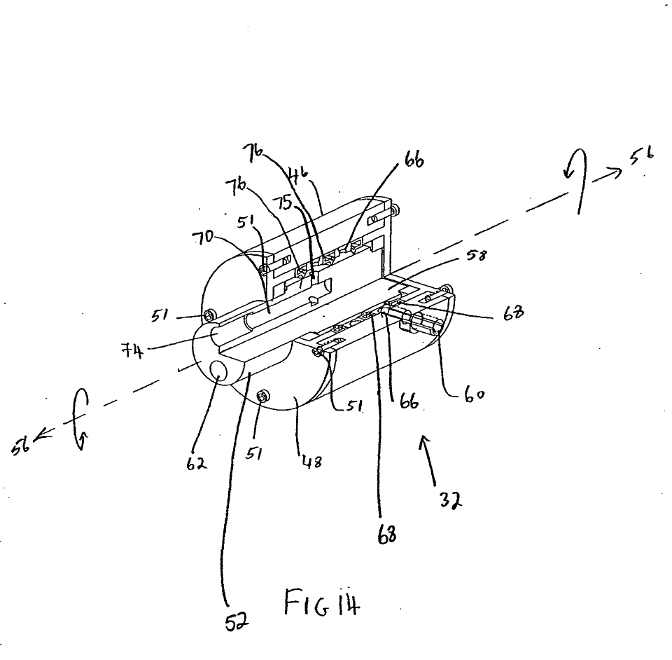

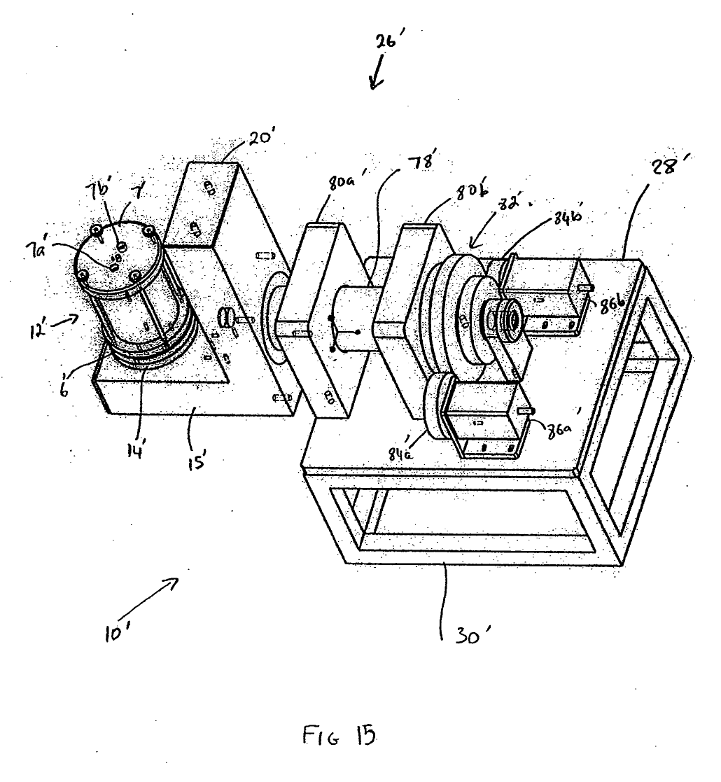

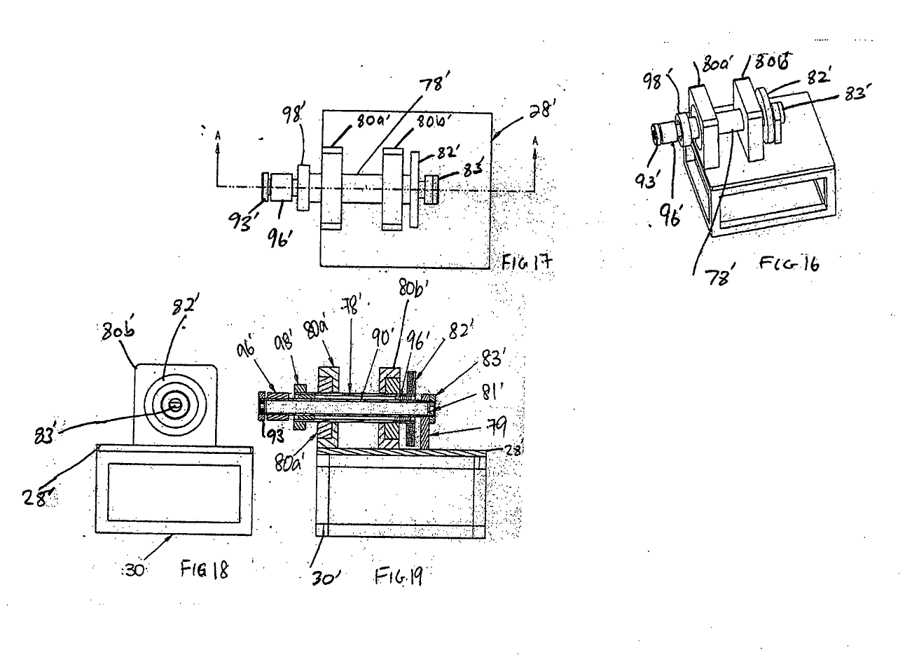

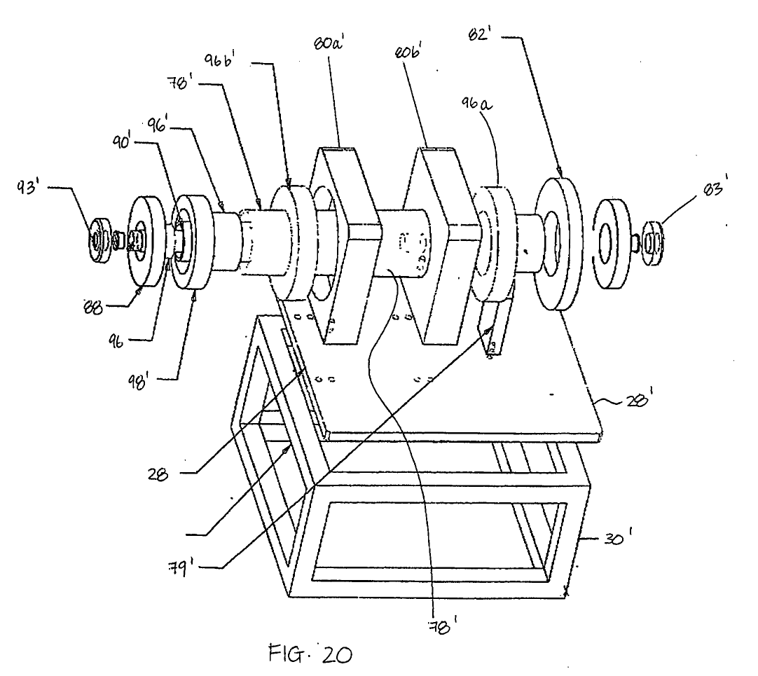

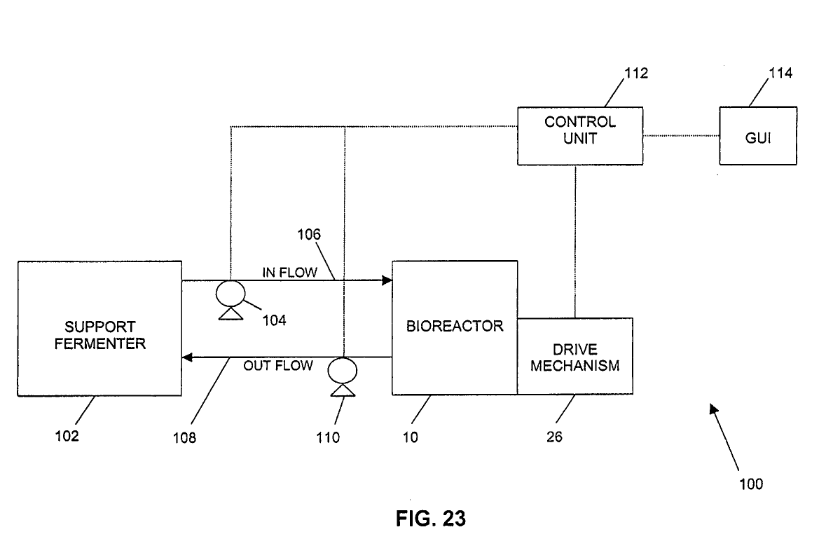

FIGS. 16-20 show the drive mechanism 26′ of bioreactor 10′ in greater detail. The drive mechanism 26′ includes a main drive shaft 78′, which extends through two mounting plates 80a′,80b′ attached to base plate 28′. The drive shaft 78′ extends through the mounting plate 80a′ and connects to the rotary L-shaped bracket 20′ to rotate the arm in use. A gear train 82′ is provided adjacent to mounting plate 80b′ and is driven by rotors 84a′,84b′ that are respectively actuated in use by the servo motors 86a′,86b′ mounted on base plate 28′. The servo motors 86a′,86b′ are operated by a controller 112 (refer to FIG. 23), as will be described further below. The servo motor 86a′ drives the drive shaft 78′ and hence the rotary L-shaped bracket 20′. The servo motor 86b′ drives an inner shaft 90′ located within the drive shaft 78′ as shown in FIG. 20, which shows an exploded perspective view of the drive shaft 78′ and inner shaft 90′, to drive the rotary plate 14′.

Referring now to FIGS. 15 to 20, the various components that make up the drive mechanism 26′ will be explained in detail. It can be seen from FIG. 19 that the shaft 78′ also includes an inner shaft 90′, which is located inside the shaft 78′ and is coupled to the rotary drive 13′ of servo-motor 84b.

plate 14 as will be explained further below.

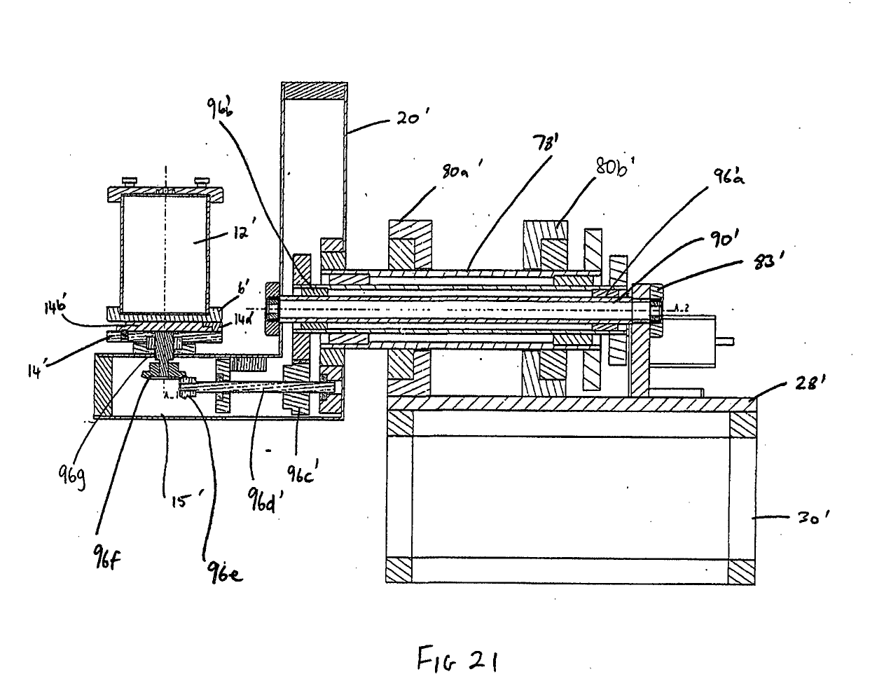

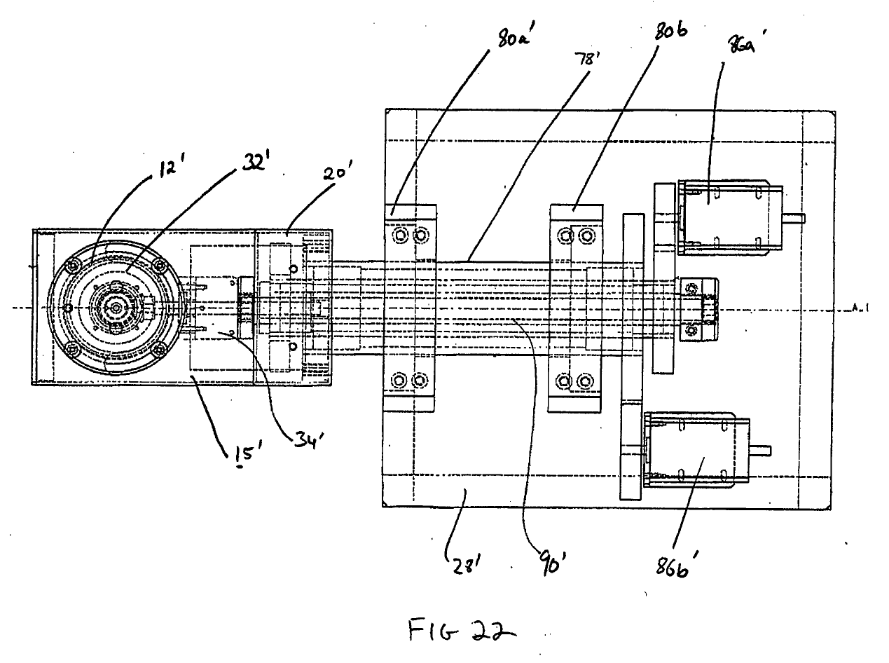

An end cap 93′ is provided at the end of the shaft. The tubes 62c′,72c′ extend through the inner shaft and are connected to the connector 34 for transport of feed material to and product material from the chamber. Another end shaft cap 83′ is provided adjacent to the gears and mounted to a tube collar 81′. A spur gear 88′ is provided on the inner shaft 90′ to allow rotary motion of the inner shaft 90′ to be transferred to gear 96c′ and through shaft 96d′, bevel gears 96e,96f for driving rotary plate 14′. A bracket intershaft 79′ is also provided to support the inner shaft 90′. A clamp lock 98′ is provided on the drive shaft 78 to transmit the rotary motion of drive shaft 78′ to the L-shaped bracket 20′. Bearings 96′ are provided on the inner shaft 90′ and the outer drive shaft 78′ to carry loads imparted by the inner shaft 90′ as it rotates in use. As shown in FIG. 21, inner shaft 90′ has a spur gear 88 for rotating with a matching spur gear 96c′ for rotating actuating rod 96d′. At the end of actuating rod 96d′ is a bevel gear 96e′ which actuates corresponding bevel gear 96f for turning shaft 96g and hence rotating plate 14′.

Referring now to FIG. 23, there is shown a schematic diagram of a system 100 for growing three-dimensional cell or tissue cultures in vitro using the bioreactor 10 or 10′. For convenience, only bioreactor 10 will be described.

The system 100 includes a support fermenter 102 into which feed material is initially supplied. A pump 104 is provided on feed line 106, which transports feed material from the support fermenter 102 to one of the pipe connectors 32,34 or both. A pump 110 is provided on product line 108, which is coupled to one of the pipe connectors 32,34 or both. The product line 108 transports product media from the bioreactor 10 to the support fermenter 102.

A controller in the form of control unit 112 is electrically connected to the pumps 104, 110 and the drive mechanism 26 of the bioreactor 10. The controller includes a pump controller which is used to control the flow rate of feed material in feed line 106 and product material in product line 108. The control unit 112 is also electrically coupled to the servo motors 86a, 86b, which respectively drive the inner shaft 90 and drive shaft 78. The control unit 112 is also coupled to temperature sensor 7f, dissolved oxygen sensor 7g, pH sensor 7h and force detector 7m, to thereby allow for a number of process variables to be monitored during use.

In use, the support fermenter 102 is initially filled with a feed material for growing cell or tissue culture. The chamber 12 is placed on the rotary plate 14 and locked thereon by the knurled locking nut and bolt arrangement 4. The pipe connectors 32,34 are then attached to the inflow pipe 106 and the outflow pipe 108 by connecting to the inlet nipples 60 and outlet nipples 72 as described above. The chamber cover 7i is removed and the chamber 12 is seeded with cell or tissues and a three dimensional matrix or a scaffold is attached to the ends of needles 7j,7k.

Prior to use, the bioreactor 10, inflow line 106, product line 108 and pipe connectors 32,34 are first cleansed and sterilized. This may be achieved by a sterilizing solution that is coupled to a valve (not shown) on the inflow line 106 and circulated through an outlet valve (not shown) on the outflow line 108.

During use, the control unit 112 activates the pumps 104 and 110 so that feed media is supplied to the bioreactor 10 and product material is transferred from the bioreactor 10, thereby causing continuous circulation between the support fermenter 102 and the bioreactor 10. At the same time, the control unit 112 activates the servo-motors 86a and 86b to respectively rotate the drive shaft 78 and the inner shaft 90. This causes the chamber 12 to rotate about the vertical axis 16 in the direction of arrow 18 while simultaneously rotating the chamber about the horizontal axis 22 in the direction of arrow 24, as shown in FIG. 1.

As the control unit 112 is coupled to servo-motors 86a,86b, it is able to control the speed of rotation of the chamber about the horizontal axis 22 and the vertical axis 18. The direction of rotation may also be altered from that shown in FIG. 1.

A control system diagram for the system 100 is shown schematically in FIG. 25. As can be seen in this diagram, the control unit 112 is provided with two digital encoders for monitoring the speed and position of the servo-motors 86a and 86b. The control unit 112 is also connected to a Graphical User Interface (GUI) 114 connected to a PC 114a, to provide a user interface for a user to control the system 100. The slip ring assemblies allow data exchange and transmission between the servo motors 86a,86b and the control unit 112 as can be seen by the heater element, and the detectors for pH, temperature and dissolved oxygen. The control unit 112 is able to operate the bioreactor 10 in three modes: manual mode, jogging mode and profile mode.

The manual mode of operation allows the user to set the rotational speed and directions of both the vertical axis 16 and the horizontal axis 22 of rotation. The preset values can be changed during operation.

The jogging mode allows the user to oscillate the rotary L-shaped bracket 20 and the chamber 12 by setting speeds and the angles of oscillation.

The profile mode allows the user to set up to twenty settings of speed, time and direction for the operating variables of the bioreactor 10. A graphical profile of the execution of the settings can be shown graphically on the GUI 114. The bioreactor 10 can also be programmed in this mode to operate the settings in a continuous loop.

An advantage of the bioreactor of the present invention is that stable cell culture conditions can be achieved in the bioreactor system 100 throughout the course of cell culture growth. Experiments have been conducted to affirm this. The table below illustrates the average daily dissolved oxygen reading, pH reading and temperature reading in chamber 12 of bioreactor 10 for a period of 15 days when fluid material was re-circulated between reactor 10 and the support fermenter 102.

TABLE 1
Rotary L-arm bracket (20) speed: 3 to 30 rpm
Rotary plate (14) speed: 3 to 30 rpm

| Day | DO Reading | pH Reading | Temperature Reading (° C.) |
|---|---|---|---|
| 1 | 26.7 | 7.4 | 37 |
| 2 | 26.0 | 7.4 | 37 |
| 3 | 26.0 | 7.4 | 37 |
| 4 | 25.8 | 7.4 | 37 |
| 5 | 25.6 | 7.4 | 37 |
| 6 | 26.0 | 7.4 | 37 |
| 7 | 26.3 | 7.4 | 37 |
| 8 | 26.4 | 7.37 | 37 |
| 9 | 25.7 | 7.37 | 37 |
| 10 | 26.2 | 7.37 | 37 |
| 11 | 25.2 | 7.37 | 37 |
| 12 | 27.3 | 7.4 | 37 |
| 13 | 25.5 | 7.4 | 37 |
| 14 | 25.9 | 7.4 | 37 |
| 15 | 26.7 | 7.4 | 37 |

As can be seen from the above results, constant dissolved oxygen level, pH and temperature are maintained throughout the 15 days.

By providing very stable oxygen, pH and temperature conditions, it is possible to mimic the physiological conditions of cells and tissues.

As the L-shaped bracket 20 and the rotary plate 14 are couple to respective servo-motors 86a,86b, the flow regimes within the bioreactor can be altered. This is achieved by being the to varying the speed of either the L-shaped bracket 20 or the rotary plate 14 so that the chamber 12 rotated at different speeds along either the horizontal axis 22 or verticle axis 16. If the speeds of rotation along either the horizontal axis 22 or vertical axes were fixed with respect to each other, the flow regimes within the chamber 12 would be fixed according to the single speed.

As flow regimes within the chamber 12 can be altered by independently varying the speeds of the L-shaped bracket 20 and the rotary plate 14, it is possible to dynamically optimize the conditions within the chamber 12 according to the type of cells or tissues being grown. Accordingly, the bioreactor 10 can be used for research applications for determining optimal operating parameters for the growth of particular cell or tissue cultures.

The ability to dynamically change the flow regimes within the chamber 12, ensures a homogenous body of nutrients are constantly being supplied to fibroblast cells as they grow on the scaffold. Furthermore, as two force vectors or flow vectors are applied to a growing cell or tissue culture at any point in time, spent nutrients from culture media is constantly being replaced at the sites of the growing cells or tissues with fresh nutrient culture media. This is a particular advantage in three-dimensional cell and tissue cultures as the fresh nutrient culture media is able to penetrate deep within the three-dimensional structure.

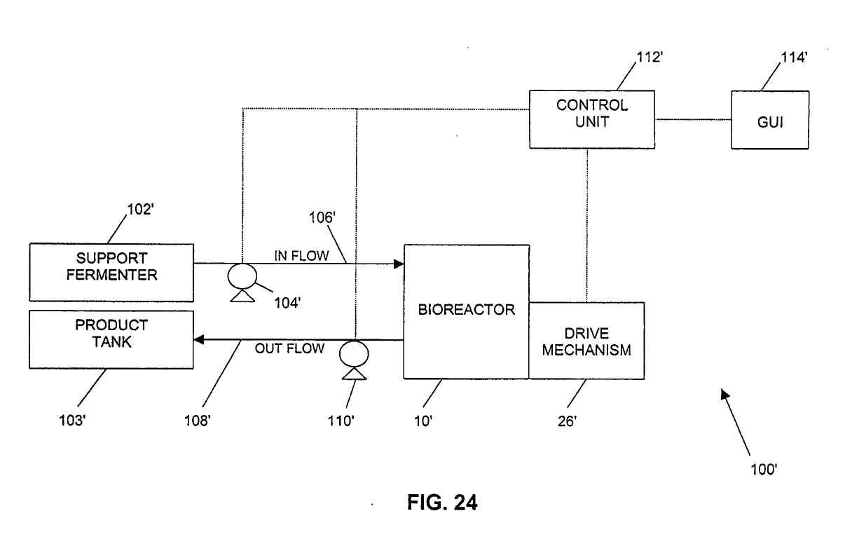

Referring now to FIG. 24, there is shown a schematic diagram of an alternative system 100′ for growing three-dimensional cell or tissue cultures in vitro using the bioreactor 10′. The unit operations of the system 100′ are the same as the unit operations of system 100 but are shown with the prime symbol (′). The difference in the system 100′ is that the product material from the bioreactor 10′ is not re-circulated back to a support fermenter 102′ but is removed from the bioreactor 10 via product line 108′ to product tank 103′. Accordingly, system 100′ shows a continuous flow bioreactor system for growing three-dimensional cell or tissue cultures in vitro.

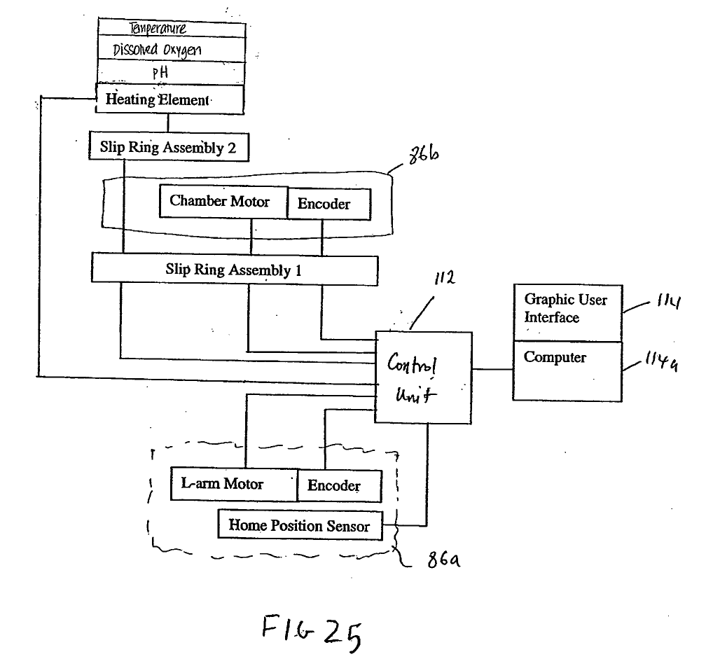

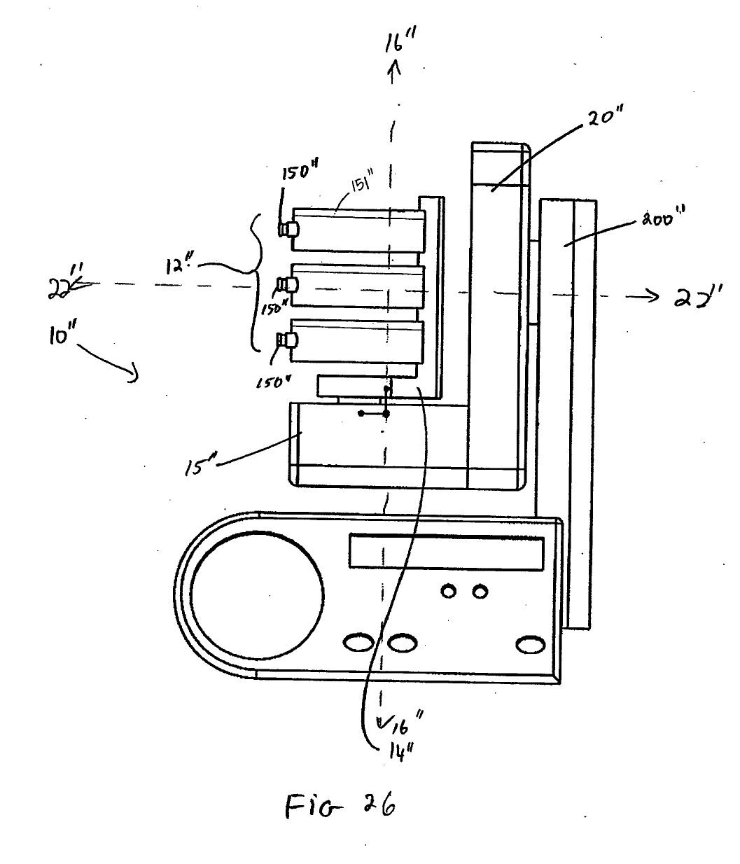

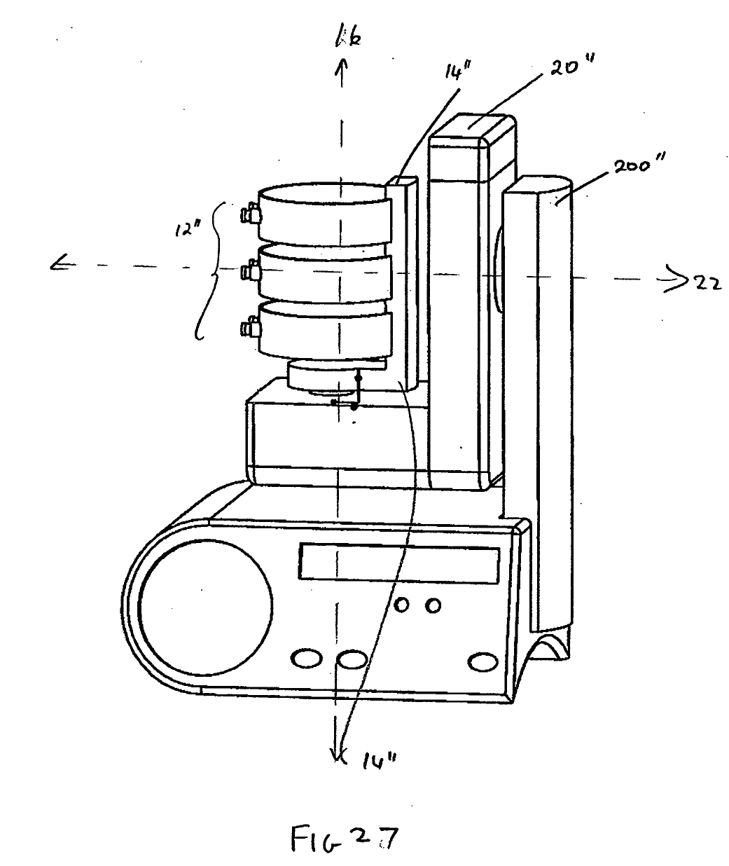

Referring now to FIGS. 26 to 28, there is shown a third alternative embodiment of a dual axis bioreactor 10″ for growing cell or tissue cultures. The bioreactor 10″ includes a cell or tissue culture modules 12″ made of polycarbonate and constructed with a thin silicon membrane on one side for gas exchange within an incubator in which the bioreactor 10″ is placed.

The cell or tissue culture modules 12″ include a cap (151″) which is removed for allowing a user to place nutrient medium into the modules 12″ for growing cell or tissue cultures on a scaffold. In this embodiment, the scaffold is fixed to a mount in the form of two surgical needles (not shown) which are fixed to the inside of each module 12″. The bioreactor 10″ can be placed within a CO₂ incubator so that the thin silicon membrane on the side of the capsule allows ingress of CO₂ to thereby produce a HCO₃^{-/CO}₂ system, which acts as a buffer to maintain the pH of the culture media.

In this embodiment, the modules 12″ are rotated about the vertical axis 16″ by L-bracket 14″ that is coupled to L-bracket 20″ which rotates about horizontal axis 22″. The L-bracket 20″ is mounted on stationary frame 200″. Two servo-motors (not shown) can rotate the L-brackets 14″,20″ about their respective axes and a drive mechanism and control system (not shown) similar to the drive mechanisms 26,26′ and control unit 112 could be used to operate the bioreactor 10″ as will be understood by persons skilled in the art.

It should be realized that the bioreactors 10,10′, 10″ of the present invention can be used to grow any type of cell or tissue and is advantageously can be used to grow three-dimensional cell or tissue culture for the formation of tissues including, but not limited to, skin; bone marrow; liver, pancreas, kidney, adrenal and neurological tissues.

The examples described herein illustrates the various uses of the bioreactor 10.

### EXAMPLES

### EXAMPLE 1

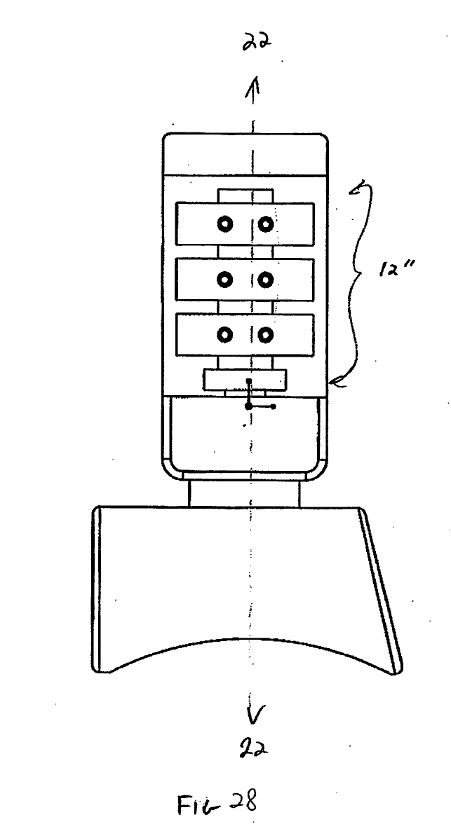

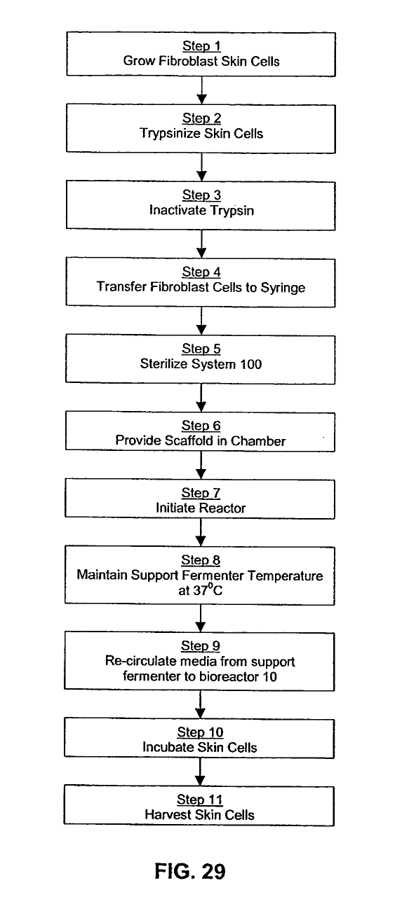

### Three-Dimensional Skin Culture preparation FIG. 29 schematically shows the steps of a method that was used to grow a three-dimensional skin culture in vitro using the system 100 as follows:

Step 1: Human fibroblast skin cells were grown to confluency in a 150 cm² Falcon tissue culture flask containing 20 ml. of a culture medium consisting of Dulbecco's modified Minimum Essential Medium (MEM) containing 10% fetal calf serum. Dulbecco's modified Minimum Essential Medium is a standard commercially available culture medium obtained from Microbiological Associates, Bethesda, Md., United States of America.

Step 2: The spent culture medium was removed from the flask and the fibroblast cell growth was trypsinized with 2 ml of 0.25% trypsin in phosphate buffered saline for three minutes.

Step 3: The trypsin was inactivated by dilution with a 20 ml portion of the same culture medium.

Step 4: The fibroblast cells were then transferred to a sterile syringe.

Step 5: The chamber 12 of the bioreactor 10, the feed line 106, the product line 108, the pipe connectors 32,34 and the support fermenter were gas sterilized with ethylene oxide, washed with sterile water to remove ethylene oxide residue and then equilibrated by priming with Dulbecco's modified Minimum Essential Medium (MEM) containing 2% fetal calf serum.

Step 6: A nylon polyester fiber scaffold cylinder having a diameter of 80 mm and a height of 180 mm was provided in the chamber 12. The chamber 12 was inoculated with 30 ml of the fibroblast cell suspension in the syringe of step 4 to begin incubation of the fibroblast cells.

Step 7: Using the control unit 112, bioreactor 12 was activated to rotate the chamber 12 about the vertical axis 16 in the direction of arrow 18 at 20 rpm and the horizontal axis 22 in the direction of arrow 24 at 20 rpm.

Step 8: The media within the support fermenter was maintained at a temperature of 37° C. by a water bath surrounding support fermenter.

Step 9: After the first hour of incubation, pumps 104 and 110 re-circulated the media of the chamber 12 from the support fermenter 102 to the chamber 12 to maintain the temperature of the media during incubation.

Step 10: The chamber 12 was allowed to incubate to grow skin cells for 3 days.

Step 11: At the termination of incubation, the skin cells were harvested by removing the scaffold from the chamber 12 and thoroughly washing the chamber 12 in saline.

The scaffold contained three-dimensional skin tissue. The skin fibroblasts had stretched across the mesh openings. The skin cells had cell-cell and cell-matrix interactions that were characteristic of whole tissue in vivo cells. The three-dimensional skin tissue can be cut and used in a variety of applications.

Preparation of Media and Reagents

The following reagents in examples 2 to 6 were prepared as follows: Preparation of DMEM+F12 Media, Required Volume: 1000 ml 1. Measure out 80% of the required volume or 800 ml of ultrapure water.

2. Add DMEM+F12 media powder to the ultrapure water and stir gently.

3. Add 16.0 ml of 7.5% w/v sodium bicarbonate solution.

4. Adjust pH of the media to 0.1-0.3 units below the desired pH of 6.8. 5. Top up with ultrapure water to the required volume of 1000 ml.

6. Sterilize immediately by membrane filtration using a membrane with porosity of 0.22 μm.

7. Aseptically disperse the media into a sterilized bottle.

8. Aliquot out a small volume into a centrifuge tube and incubate it for a sterility check.

9. Store the remaining media in a fridge at 4° C.

10. Complete the media by adding 10% FCS w/v and 1% Pen/Strep/Amp w/v, after it has pass the sterility check.

Preparation of Collagenase II

1. Dissolve completely 0.1 g of collagenase II powder into 50 ml of serum-free media.

2. Filter the solution through a 0.22 μm filter disc.

3. Disperse the solution into centrifuge tubes and store them at 4° C.

Preparation of Sodium Alginate

1. Dissolve completely 1.5 g of sodium alginate into 100 ml of PBS solution.

2. Autoclave the solution or filter it with 1.8 μm filter disc for at least 3 times.

Preparation of PBS solution [10× stock]

1. Dissolve the following components in 1000 ml of ultrapure water:

| | |
|---|---|
| NaCl | 80 g |
| KCl | 2 g |
| KH₂PO₄ | 2 g |
| Na₂HPO₄.2H₂O | 14.1 g |

2. Sterilization of PBS is done by autoclaving a 1×PBS stock.

### EXAMPLE 2

### Isolation of Chondrocytes/Cartilage from Pig's Ears

Step 1: Surface sterilization was conducted on the pig's ears in a bio-safety cabinet. Three beakers were filled with iodine, alcohol and PBS respectively. The pig's ears are then soaked in each beaker for 15 minutes.

Step 2: The ears were placed on a sterile plate and the skin and other muscle tissues removed leaving behind only the cartilage.

Step 3: The cartilage was transferred onto a new sterile plate and cut into thin slices. This facilitates digestion at a later stage. A small amount of PBS was added to keep the cartilage wet. The thin slices of cartilage were then aseptically transferred into 50 ml centrifuge tubes.

Step 4: Collagenase II was added into the centrifuge tubes to form a cell suspension. The tubes were placed into a shaking incubator for 16-18 hrs at 37° C. to ensure homogenous digestion. Digested cartilage is indicated by a change in color of the collagenase II from red to yellow with turbidity.

Step 5: A little of the digested cartilage was removed and tested for contamination using an inverted microscope.

Step 6: The cell suspension is then filtered through a sterile nylon filter to remove any undigested cartilage.

Step 7: 20 ml of PBS was added to the filtered cell suspension, and the resulting mixture centrifuged at 2500 rpm for 5 min.

Step 8: The supernatant resulting from the centrifuge was carefully poured away and the residual cartilage (also known as chondrocytes) was washed with PBS to remove the collagenase II.

Step 9: The centrifuge tubes containing the washed cartilage was inverted and centrifuged at 2500 rpm for 3 minutes. Thereafter, the PBS was removed from the centrifuge tubes.

Step 10: 10 ml DMEM media was added to the cartilage, followed by a transfer into a T-25 flask to check for contamination under a microscope.

Step 11: The isolated cartilage tissue was then placed in the bioreactor 10. Conditions therein are at a temperature of 37° C. and 5% volume CO₂.

A three-dimensional cartilage tissue was obtained in which cartilage tissue had cell-cell and cell-matrix interactions that were characteristic of in vivo cartilage tissue.

### EXAMPLE 3

### Thawing and Maintenance of Cells

Step 1: A cryovial of Goat Chondrocyte cells was removed from liquid nitrogen and placed them immediately into a water bath set at 37° C. for less than 1 minute until the last trace of ice vanishes.

Step 2: The cryovial was then removed from the water bath and sprayed with 70% ethanol before placing it in the biosafety cabinet.

Step 3: The cryovial was then transferred into a centrifuge tube containing 9 ml sterile DMEM media and spun at 1500 rpm for 6 minutes.

Step 4: After centrifugation, the supernatant was removed and the residual cryovial was re-suspended in 2 ml sterile DMEM media. About 1511 of the suspension is then removed for analysis on the number of viable cells count.

Step 5: More than 1×10⁵ cells/ml were then seeded into a T-75 flask with 20 ml sterile DMEM media, and the cells were incubated in the bioreactor 10 at 37° C., 5% CO₂.

Step 6: Cell growth was examined daily and replenished with fresh DMEM every 3 days.

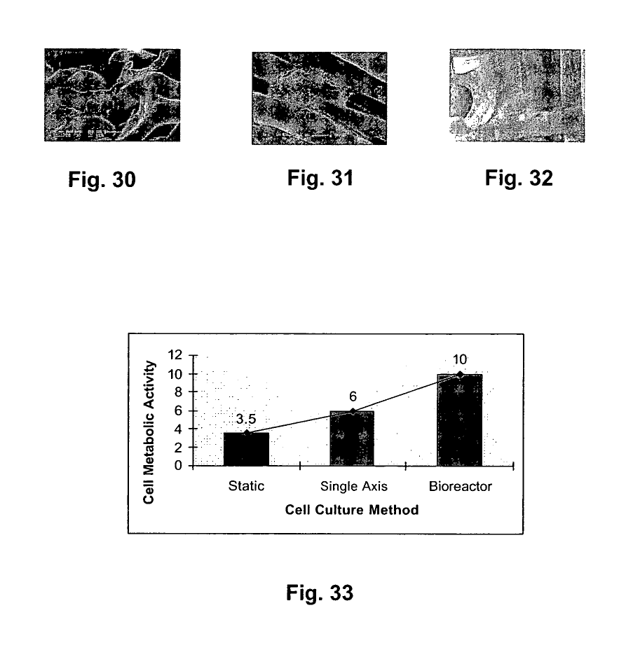

FIGS. 30 to 32 shows a SEM micrograph of goat chondrocytes seeded onto a 3D ear shaped scaffold. The cultured chondrocytes of FIG. 30 were incubated in a static environment, the cultured chondrocytes of FIG. 31 were incubated in a bioreactor that was subjected to a single a single axis of rotation and the cultured chondrocytes of FIG. 32 were incubated in the bioreactor 10 which subjected the cells to axes of rotation.

From FIG. 32, it can be seen that the scaffold cultured in the bioreactor 10 of the present invention, contained three-dimensional skin tissue in which the skin fibroblasts had stretched across the mesh openings of the scaffold. The skin cells had cell-cell and cell-scaffold interactions that were characteristic of whole cartilage tissue in vivo cells.

In comparison with FIG. 30, the ear shaped scaffold cultured in the static environment has hardly any skin tissues forming therein.

Further in comparison with FIG. 31, the ear shaped scaffold cultured in a single axis rotating reactor, although has more skin tissue forming as compared to that in FIG. 30, is still not as fully developed as that in FIG. 32.

FIG. 33 illustrates cell metabolic activity according to each of the three environments—static environment, single axis rotating reactor and the bioreactor 10. As can be clearly seen, the cell metabolic activity is highest in the bioreactor 10, followed by the single axis rotating reactor and lowest in the static environment. This indicates that the bioreactor 10 cultures tissue having cell-cell and cell-matrix interaction.

### EXAMPLE 4

### Expansion of Cells

Step 1: Within the biosafety cabinet, spent media in a culture flask was pipetted out.

Step 2: 6 ml of trypsin was pipetted into the culture flask to dislodge the cells and the flask was incubated in the bioreactor at 37° C., 5% CO₂.

Step 3: Once all cells are detached from the flask, 12 ml of DMEM media was added into the culture flask to stop trypsinisation.

Step 4: The contents in the culture flask were pipetted into a centrifuge tube and sent for centrifugation at 1000 rpm for 10 minutes.

Step 5: After centrifugation, the supernatant was removed and the residual cells were re-suspended in 5-10 ml of complete DMEM (Sterile) media.

Step 6: 15 μl of cell suspension was aliqouted out for cell counting and determining the total cell number.

Step 6: The cells were sub-cultured into many culture flasks with a specified cell density.

Step 7: The culture flasks are then incubated in the bioreactor 10 at 37° C., 5% CO₂.

### EXAMPLE 5

### Preparation of a Scaffold and Seeding of the Scaffold

Step 1: In a biosafety hazard hood, scaffolds are placed into a sterile beaker. Ethanol was added to fill the entire beaker and left alone for 12 hrs.

Step 2: The ethanol was removed after 12 hrs and sterile PBS added to fill the entire beaker and left to stand for another 12 hrs.

Step 3: After 12 hrs, the PBS was removed and the scaffolds were dried by leaving them in the biosafety hazard hood for another 12 hrs

Step 4: Within the biosafety cabinet, spent media in the culture flask were pipetted out. 6 ml of trypsin was pipetted into the culture flask to dislodge the cells.

Step 5: The flask was incubated at 37° C., 5% CO₂ in the bioreactor 10.

Step 6: Once all cells are detached from the flask, add 12 ml of complete DMEM (Sterile) into the culture flask to stop trypsinisation.

Step 7: The contents in the culture flask were pipetted into a centrifuge tube and send for centrifugation at 1000 rpm for 10 minutes.

Step 8: After centrifugation, the supernatant was removed and the residual cells re-suspended in 10 ml of DMEM media.

Step 9: The cell suspension is mixed with twice the amount of sterile sodium alginate solution to obtain a homogenous cell suspension. (4 mls of cell suspension per scaffold)

Step 10: The scaffolds were soak in sterile calcium chloride solution for a minute or so before it is seeded with the cells.

Step 11: While the scaffold is still dripping wet with the calcium chloride solution, 4 mls of the cell suspension with the sodium alginate is drawn and slowly seeded onto the scaffold. Any runoff is immediately sucked up onto the pipette and re-seeded onto the scaffold.

Step 12: After the 4 mls of cell suspension is seeded onto the scaffold, the scaffold is once again soaked in calcium chloride solution for a few seconds to make sure all the sodium alginate is coagulated to form a gel.

Step 13: The whole scaffold with the cells seeded is place in culture container with the media needed and incubated in the bioreactor 10 respectively.

### EXAMPLE 6

### Different Types of Assays MTS Assay

Step 1: Drain the medium from the wells containing the scaffolds and add 500 μl of fresh serum free basal medium into the wells.

Step 2: The plates were to be wrapped immediately in aluminium foil to avoid any light exposure.

Step 3: Add 100 μl of MTS reagent into each well.

Step 4: Incubate for 3 hrs in the 5% carbon dioxide bioreactor 10.

Step 5: After incubation, pipette the content of the wells to get a homogenous mixture and add 100 μl of the homogenized suspension into a 96 well culture plate.

Step 6: Read the sample using a plate reader at a wavelength of 490 nm and calculate the mean value to obtain the result.

FDA and PI Viability Assay

Step 1: A Propidium Iodide Stock Solution [10 mg/ml PI in PBS] is prepared by diluting 100 ml stock solution in 1 ml PBS.

Step 2: A Fluorescein Diacetate Stock Solution [5 mg/ml FDA in PBS], is prepared by diluting 40 ml stock solution in 10 ml PBS.

Step 3: The samples were washed with PBS for 2 times.

Step 4: Samples were incubated at 37° C. with FDA working solution for 15 minutes.

Step 5: FDA working solution was removed and rinsed the sample twice with PBS.

Step 6: PI working solution was added into each sample, making sure the solution had covered the entire sample and incubated for 2 minutes at room temperature.

Step 7: Samples were rinsed twice again with PBS and viewed under the Fluorescent Microscope.

### INDUSTRIAL APPLICATIONS

It will be appreciated that the cell and tissues grown/incubated by the bioreactor, system and method disclosed above can be used to prepare three-dimensional tissues and two-dimensional tissues, neo-tissue, a suspension of cells, scaffold constructs, and neo-tissue constructs.

The bioreactor, system and method disclosed herein provide physical signaling in two force vectors to grow three-dimensional cell or tissue cultures that mimic the function and structure of the parent tissue. The three-dimensional cell or tissue cultures of the present invention show superior characteristics over tissues grown by a single force vector.

Without being bound by theory, it is though that by applying two force vectors during incubation, the culture medium penetrates into the pores of any three dimensional matrices on which the cell or tissue cultures are grown. This enhanced penetration and induces a more penetrating flow pattern through the three-dimensional matrix, allowing the medium to reach fibroblast cells in the center of the matrix.

The pipe connectors of the present invention provide the advantage of allowing the medium to be re-circulated between the bioreactor and a support fermenter or some other unit operation in an industrial process without entanglement of any attached pipes as the reactor rotates. This allows the bioreactor to operate continuously, thereby achieving greater efficiencies that could not be achieved with a batch operated bioreactor.

Another advantage of the pipe connectors is that they allow the flow of multiple streams into and out of the reactor.

The bioreactor of the present invention provides a device for growing cells that have cell-cell and cell-matrix interactions that are characteristic of whole tissue in vivo cells grown in three-dimensions.

The three-dimensional culture tissues produced by the bioreactor, system and method of the invention can be used in a variety of applications, including, not limited to, transplantation or implantation of either the cultured cells obtained from the matrix, or the cultured matrix itself in vivo. For transplantation or implantation in vivo, either the cells obtained from the culture or the entire three-dimensional culture could be implanted, depending upon the type of tissue involved. For example, three-dimensional bone marrow cultures can be maintained in vitro for long periods; the cells isolated from these cultures can be used in transplantation or the entire culture may be implanted. By contrast, in skin cultures, the entire three-dimensional culture can be grafted in vivo for treating burn victims, skin ulcerations and wounds.

Three-dimensional tissue culture implants may, according to the invention, be used to replace or augment existing tissue, to introduce new or altered tissue, to modify artificial prostheses, or to join together biological tissues or structures. Examples include: three-dimensional bone marrow culture implants for replacing bone marrow; three-dimensional liver tissue implants used to augment liver function; hip prostheses coated with three-dimensional cultures of cartilage; and dental prostheses joined to a three-dimensional culture of oral mucosa.

The bioreactor of the present invention can be used to reproducibly create uniform tissues with suitable biochemical and mechanical properties. The bioreactor could be used for research applications, where one or a small number of cells or tissue constructs are made by an individual researcher, or on an industrial scale to meet market demand.

It will be appreciated that the bioreactor of the present invention ensures a constant removal of metabolic waste products and provides the growing tissue with a constant supply of fresh nutrients.

The bioreactor of the present invention grows cells and tissues that do not loose their differentiation status and are therefore functionally similar. Furthermore, the cells can be multiplied in a more natural way by culturing them in a bioreactor system which closely mimics the conditions of a naturally occurring physiological system.

The ability to dynamically control the speed at which the chamber of the bioreactor rotates about both horizontal and vertical axes allows physiologic tissue remodeling whereby the optimal parameters of incubation can be determined. It also provides a constant and regulatory supply of nutrition to the growing cells or tissues and a system for removal of metabolic byproducts. The bioreactor also maintains an organotypic environment to maintain cellular differentiation and optimal function.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A dual axis bioreactor for growing cell or tissue cultures comprising:
a chamber for containing a cell or tissue culture and a culture medium for growing cell or tissue cultures;
a drive mechanism for rotating the chamber at a first speed about a first axis and at a second speed about a second axis, the second axis being substantially normal relative to the first axis, wherein the magnitude of the first speed and the second speed are independently variable of each other to thereby grow a cell or tissue culture within the chamber.

2. A dual axis bioreactor as claimed in claim 1, further comprising:
a first rotatable member rotatable about the first axis and coupled to the chamber for rotating the chamber about the first axis; and
a second rotatable member rotatable about the second axis, the second rotatable member coupled to the chamber for rotating the chamber about the second axis.

3. A dual axis bioreactor as claimed in claim 1, further comprising at least one fluid connector comprising:
a stationary casing;
a rotatable shaft mounted to the stationary casing, the shaft rotatable about a shaft axis in axial alignment with, or axially offset from, either the first or second axes; and
at least one fluidly sealed passage defined between the juncture of the stationary casing and the rotatable shaft and extending through the casing and the rotatable shaft, wherein the fluidly sealed passage allows passage of fluid from or to the chamber, or both, as the shaft rotates about the shaft axis.

4. A dual axis bioreactor as claimed in claim 1, further comprising a heater element that is thermally coupled to the chamber for heating material within the chamber.

5. A dual axis bioreactor as claimed in claim 4, wherein the heater element is disposed adjacent to an outer surface of the chamber.

6. A dual axis bioreactor as claimed in claim 1, further comprising one or more detector elements for detecting a variable of the material within the chamber.

7. A dual axis bioreactor as claimed in claim 6, wherein the variable of the material within the chamber is selected from the group consisting of: pH; temperature; dissolved oxygen content; and one or more combinations thereof.

8. A dual axis bioreactor as claimed in claim 1, further comprising a force detector for detecting the force applied the chamber as it rotates about the first axis or the second axis, or both.

9. A dual axis bioreactor as claimed in claim 3, wherein one fluidly sealed passage is provided in the fluid connector for passage of feed material to the chamber, and another fluidly sealed passage is provided in the fluid connector for passage of product material from the chamber.

10. A dual axis bioreactor as claimed in claim 2, further comprising an adjustment mechanism provided on the first rotatable member or the second rotatable member for respectively adjusting the position of the chamber relative to the second axis or the first axis.

11. A dual axis bioreactor as claimed in claim 2, wherein the drive mechanism includes at least one motor that is coupled to the first or second rotatable members, or both, by at least one drive shaft.

12. A dual axis bioreactor as claimed in claim 2, wherein the drive mechanism includes:
a first motor coupled to the first rotatable member by an outer drive shaft having a hollow passage extending through its axis; and
a second motor coupled to the second rotatable member by an inner drive shaft disposed at least partly within the hollow passage of the outer drive shaft.

13. A dual axis bioreactor as claimed in claim 2, wherein the drive mechanism includes:
a first motor coupled to the first rotatable member by a first drive shaft; and
a second motor disposed within, or on, the second rotatable member and coupled to the second rotatable member by a second drive shaft.

14. A dual axis bioreactor as claimed in claim 11, wherein the first and second motors are servo motors.

15. A dual axis bioreactor as claimed in claim 11, wherein the drive shaft is coupled to the motor by a gear train for controlling the speed of rotation of the shaft.

16. A dual axis bioreactor as claimed in claim 1, wherein the chamber further comprises a feed conduit for passage of feed media into the chamber and an outlet conduit for passage of product material from the chamber.

17. A method for growing cell or tissue cultures in vitro comprising the steps of:
(a) providing a chamber having a cell or tissue culture and a culture medium;
(b) rotating the chamber about a first axis at a first speed; and
(c) rotating the chamber about a second axis at a second speed, the second axis being substantially normal to the first axis and wherein the magnitude of the first speed and the second speed are independently variable of each other to thereby grow a cell or tissue culture.

18. A system for growing cell or tissue cultures in vitro comprising:
a bioreactor comprising
a chamber for containing a cell or tissue culture and a culture medium for growing cell or tissue cultures;
a drive mechanism for rotating the chamber at a first speed about a first axis and at a second speed about a second axis, the second axis being substantially normal relative to the first axis; and
a controller for controlling the operation of the drive mechanism, wherein the magnitude of the first speed and the second speed are independently variable of each other to thereby grow a cell or tissue culture within the chamber.

19. A continuous flow dual axis bioreactor for growing cell or tissue cultures comprising:
a chamber for containing a cell or tissue culture and a culture medium;
a first rotatable member rotatable about a first axis, the first rotatable member coupled to the chamber for rotating the chamber about the first axis in use;
a second rotatable member rotatable about a second axis, the second axis being substantially normal relative to the first axis, the second rotatable member coupled to the chamber for rotating the chamber about the second axis;
a drive mechanism for rotating the first rotatable member at a first speed about the first axis and the second rotatable member at a second speed about the second axis, wherein the magnitude of the first speed and the second speed are independently variable of each other to thereby grow a cell or tissue culture within the chamber; and
a fluid connector for providing fluid material passage to and from the chamber.

20. A continuous flow dual axis bioreactor as claimed in claim 19, wherein the fluid connector comprises:
a stationary casing;
a rotatable shaft mounted to the stationary casing, the shaft rotatable about a shaft axis in axial alignment with, or axially offset from, either the first or second axes; and
at least two fluidly sealed passages defined between the juncture of the casing and the rotatable shaft and extending through the casing and the rotatable shaft, wherein both fluidly sealed passages are configured such that they are in fluid communication with the chamber as it rotates about the first and second axes;
wherein in use, one of the fluidly sealed passages provides an inlet passage for fluid material to the chamber and the other fluidly sealed passage provides an outlet passage for removal of fluid material from the chamber.

21. A cell or tissue culture when grown in vitro by the method of claim 17.

22. A three-dimensional cell or tissue culture when grown in vitro by the method of claim 17.

23. A dual axis bioreactor as claimed in claim 1, wherein the chamber further comprises a mount for retaining a scaffold for growing three-dimensional tissue culture constructs.
